(19) United States
(12) Patent Application Publication
(10) Pub. No.: US 2026/0022373 A1
(43) Pub. Date: Jan. 22, 2026

(54) METHODS FOR MESSENGER RNA TAILING

(71) Applicant: SANOFI PASTEUR INC., Swiftwater, PA (US)

(72) Inventors: Yanhua YAN, Cambridge, MA (US); Zun LIU, Cambridge, MA (US)

(21) Appl. No.: 19/196,342

(22) Filed: May 1, 2025

Related U.S. Application Data
(63) Continuation of application No. PCT/EP2023/080722, filed on Nov. 3, 2023.

(30) Foreign Application Priority Data
Nov. 4, 2022 (EP) 22306661.4

(51) Int. Cl. C12N 15/11; A61K 31/7105; C12N 9/12; C12N 15/63; C12Q 1/68

(52) U.S. Cl. CPC C12N 15/11; A61K 31/7105; C12N 9/1247; C12N 15/63; C12Q 1/68; C12Y 207/07006; C12N 2830/001

(57) ABSTRACT

Provided herein is a messenger RNA (mRNA) comprising, from 5′ to 3′, a 5′ untranslated region (5′ UTR), at least one open reading frame (ORF), a 3′ untranslated region (3′ UTR), and a GC-rich sequence, wherein the mRNA comprises at least one chemical modification. Also provided are methods of producing a plurality of chemically modified mRNA molecules with polyA sequence lengths of at least about 200 consecutive adenosine nucleotides.

### RELATED APPLICATIONS

This application is a continuation of International Patent Application No. PCT/EP2023/080722, filed Nov. 3, 2023, which claims priority to European Patent Application No. 22306661.4, filed Nov. 4, 2022, the disclosures of which are hereby incorporated by reference in their entirety.

### REFERENCE TO SEQUENCE LISTING SUBMITTED ELECTRONICALLY

The instant application contains a Sequence Listing which has been submitted electronically in XML format and is hereby incorporated by reference in its entirety. Said XML file, created on May 1, 2025, is named 762944_SA9-332PCCON_ST26.xml and is 24,086 bytes in size.

### BACKGROUND OF THE DISCLOSURE

Messenger RNA (mRNA)-based therapeutics are an emerging therapeutic modality for the treatment of numerous diseases. Typically comprising, from 5′ to 3′, the elements of a 5′ cap, 5′ untranslated region (UTR), an open reading frame (ORF) encoding a polypeptide, a 3′ UTR, and a polyA tail, each element plays a role promoting expression and stability of the mRNA. Moreover, the use of chemically modified nucleotides in the mRNA may reduce the immunogenicity of the molecule. However, the enzymatic polyA tailing of chemically modified mRNA yields highly variable polyA tail lengths.

Accordingly, there exists a need to produce chemically modified mRNA with more uniform polyA tail lengths.

### SUMMARY OF THE DISCLOSURE

Provided herein is a messenger RNA (mRNA) comprising, from 5′ to 3′, a 5′ untranslated region (5′ UTR), at least one open reading frame (ORF), a 3′ untranslated region (3′ UTR), and a GC-rich sequence, wherein the mRNA comprises at least one chemical modification. Also provided are methods of producing a plurality of chemically modified mRNA molecules with polyA sequence lengths of at least about 200 consecutive adenosine nucleotides.

In one aspect, the disclosure provides a messenger RNA (mRNA) comprising, from 5′ to 3′, a 5′ untranslated region (5′ UTR), at least one open reading frame (ORF), a 3′ untranslated region (3′ UTR), and a GC-rich sequence, wherein the mRNA comprises at least one chemical modification.

In another aspect, the disclosure provides a messenger RNA (mRNA) comprising, from 5′ to 3′, a 5′ untranslated region (5′ UTR), at least one open reading frame (ORF), a 3′ untranslated region (3′ UTR), and a GC-rich sequence which comprises at least about 75% G and/or C nucleotides and is at least 14 nucleotides in length, comprises CCGGUACCG, or comprises CCG, wherein the mRNA comprises at least one chemical modification.

In certain embodiments, the GC-rich sequence comprises at least about 50% G and/or C nucleotides to 100% G and/or C nucleotides.

In certain embodiments, the GC-rich sequence is 3, 4, 5, 6, 7, 8, 9, 10, 11, 12, 13, 14, 15, 16, 17, 18, 19, or 20 nucleotides in length.

In certain embodiments, the GC-rich sequence comprises at least about 70% G and/or C nucleotides.

In certain embodiments, the GC-rich sequence comprises at least about 80% G and/or C nucleotides.

In certain embodiments, the GC-rich sequence comprises at least about 80% G and/or C nucleotides and is at least 14 nucleotides in length.

In certain embodiments, the GC-rich sequence comprises 100% G and/or C nucleotides.

In certain embodiments, the GC-rich sequence comprises CCGGUACCG. In certain embodiments, the GC-rich sequence comprises CCGGUACCGCGCGC (SEQ ID NO: 1). In certain embodiments, the GC-rich sequence comprises CCGGUACCGCGCGCGUCGA (SEQ ID NO: 13). In certain embodiments, the GC-rich sequence comprises CCGGUACCGCGCGC (SEQ ID NO: 15). In certain embodiments, the GC-rich sequence comprises CCGGUACCGCGCGCCUCGA (SEQ ID NO: 18). In certain embodiments, the GC-rich sequence comprises CCGGUACCGCGCGCC (SEQ ID NO: 20). In certain embodiments, the GC-rich sequence comprises CCGGUACCGCGCGCGGAUC (SEQ ID NO: 23). In certain embodiments, the GC-rich sequence comprises CCGGUACCGCGCGCG (SEQ ID NO: 25). In certain embodiments, the GC-rich sequence comprises CCG.

In certain embodiments, the GC-rich sequence is contained within the 3′ UTR.

In certain embodiments, the GC-rich sequence is not contained within the 3′ UTR.

In certain embodiments, the chemical modification is pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, or 2′-O-methyl uridine.

In certain embodiments, the chemical modification is pseudouridine, N1-methylpseudouridine, 5-methylcytosine, 5-methoxyuridine, or a combination thereof.

In certain embodiments, the chemical modification is N1-methylpseudouridine.

In certain embodiments, the mRNA further comprises a polyA sequence.

In certain embodiments, the polyA sequence is present in the mRNA without enzymatic addition.

In certain embodiments, the polyA sequence is at least 10 consecutive adenosine nucleotides.

In certain embodiments, the polyA sequence is between 10 and 500 consecutive adenosine nucleotides.

In certain embodiments, the polyA sequence is between 80 and 300 consecutive adenosine nucleotides.

In certain embodiments, the mRNA contains a chimeric 5′ or 3′ UTR.

In certain embodiments, the mRNA encodes at least one polypeptide.

In certain embodiments, the polypeptide is a biologically active polypeptide, a therapeutic polypeptide, or an antigenic polypeptide.

In certain embodiments, the antigenic polypeptide is derived from a pathogen.

In certain embodiments, the polypeptide comprises an antibody or fragment thereof, enzyme replacement polypeptide, or genome-editing polypeptide.

In certain embodiments, the therapeutic polypeptide comprises an antibody heavy chain, an antibody light chain, an enzyme, or a cytokine.

In certain embodiments, the biologically active polypeptide comprises a genome-editing polypeptide.

In certain embodiments, the mRNA is synthesized using in vitro transcription (IVT).

In certain embodiments, the mRNA is expressed in vivo or ex vivo.

In one aspect, the disclosure provides a DNA polynucleotide comprising a nucleic acid sequence encoding the mRNA described above.

In one aspect, the disclosure provides a vector comprising the DNA polynucleotide described above.

In certain embodiments, the vector comprises at least elements a-c, from 5′ to 3′: a. an RNA polymerase promoter; b. a polynucleotide sequence encoding an ORF; and c. a polynucleotide sequence encoding a GC-rich sequence. In certain embodiments, the vector further comprises: d. a polynucleotide sequence encoding a restriction enzyme recognition site.

In certain embodiments, the vector comprises at least elements a-e, from 5′ to 3′: a. an RNA polymerase promoter; b. a polynucleotide sequence encoding a 5′ UTR; c. a polynucleotide sequence encoding an ORF; d. a polynucleotide sequence encoding a 3′ UTR; and e. a polynucleotide sequence encoding a GC-rich sequence. In certain embodiments, the vector further comprises: f. a polynucleotide sequence encoding a restriction enzyme recognition site. In certain embodiments, the vector further comprises: g. a polynucleotide sequence encoding a polyadenylation signal.

In certain embodiments, the vector lacks a polynucleotide sequence encoding a polyadenylation signal.

In certain embodiments, the vector comprises at least elements a-d, from 5′ to 3′: a. an RNA polymerase promoter; b. a polynucleotide sequence encoding a 5′ UTR; c. a polynucleotide sequence encoding an ORF; and d. a polynucleotide sequence encoding a 3′ UTR with a GC-rich sequence present at the 3′ end of the 3′UTR. In certain embodiments, the vector further comprises: c. a polynucleotide sequence encoding a restriction enzyme recognition site. In certain embodiments, the vector further comprises: f. a polynucleotide sequence encoding a polyadenylation signal.

In certain embodiments, the vector lacks a polynucleotide sequence encoding a polyadenylation signal.

In certain embodiments, the restriction enzyme recognition site comprises one or more of a BspQI recognition site, a BssHII recognition site, a SalI recognition site, a XhoI recognition site, a BamHI recognition site, and a Acc65I recognition site.

In certain embodiments, the polynucleotide sequence encoding a GC-rich sequence comprises CCGGTACCGCGCGCAAAC (SEQ ID NO: 3).

In certain embodiments, the polynucleotide sequence encoding a GC-rich sequence comprises CCGGTACCGCGCGCAAACGAAGAGC (SEQ ID NO: 26.

In certain embodiments, the polynucleotide sequence encoding a GC-rich sequence comprises CCGGTACCGCGCGCGTCGACGC (SEQ ID NO: 11).

In certain embodiments, the polynucleotide sequence encoding a GC-rich sequence comprises CCGGTACCGCGCGCGTCGA (SEQ ID NO: 12).

In certain embodiments, the polynucleotide sequence encoding a GC-rich sequence comprises CCGGTACCGCGCGCG (SEQ ID NO: 14).

In certain embodiments, the polynucleotide sequence encoding a GC-rich sequence comprises CCGGTACCGCGCGCCTCGAGGC (SEQ ID NO: 16).

In certain embodiments, the polynucleotide sequence encoding a GC-rich sequence comprises CCGGTACCGCGCGCCTCGA (SEQ ID NO: 17).

In certain embodiments, the polynucleotide sequence encoding a GC-rich sequence comprises CCGGTACCGCGCGCC (SEQ ID NO: 19).

In certain embodiments, the polynucleotide sequence encoding a GC-rich sequence comprises CCGGTACCGCGCGCGGATCCGC (SEQ ID NO: 21).

In certain embodiments, the polynucleotide sequence encoding a GC-rich sequence comprises CCGGTACCGCGCGCGGATC (SEQ ID NO: 22).

In certain embodiments, the polynucleotide sequence encoding a GC-rich sequence comprises CCGGTACCGCGCGCG (SEQ ID NO: 24).

In one aspect, the disclosure provides a host cell comprising the vector described above.

In one aspect, the disclosure provides a pharmaceutical composition comprising the mRNA described above.

In one aspect, the disclosure provides a vector comprising at least elements a-d, from 5′ to 3′: a. an RNA polymerase promoter; b. a polynucleotide sequence encoding an ORF; c. a polynucleotide sequence encoding a GC-rich sequence; and d. a polynucleotide sequence encoding a restriction enzyme recognition site, wherein the restriction enzyme recognition site comprises one or more of a BspQI recognition site, a BssHII recognition site, a SalI recognition site, a XhoI recognition site, a BamHI recognition site, and a Acc65I recognition site.

In certain embodiments, the vector comprises at least elements a-f, from 5′ to 3′: a. an RNA polymerase promoter; b. a polynucleotide sequence encoding a 5′ UTR; c. a polynucleotide sequence encoding an ORF; d. a polynucleotide sequence encoding a 3′ UTR; e. a polynucleotide sequence encoding a GC-rich sequence; and f. polynucleotide sequence encoding a restriction enzyme recognition site, wherein the restriction enzyme recognition site comprises one or more of a BspQI recognition site, a BssHII recognition site, a SalI recognition site, a XhoI recognition site, a BamHI recognition site, and a Acc65I recognition site.

In certain embodiments, the vector further comprises a polynucleotide sequence encoding a polyadenylation signal.

In certain embodiments, the vector lacks a polynucleotide sequence encoding a polyadenylation signal.

In certain embodiments, the vector lacks a polynucleotide sequence encoding a polyadenylation signal.

In certain embodiments, the polynucleotide sequence encoding a GC-rich sequence comprises CCGGTACCGCGCGCAAAC (SEQ ID NO: 3).

In certain embodiments, the polynucleotide sequence encoding a GC-rich sequence comprises CCGGTACCGCGCGCAAACGAAGAGC (SEQ ID NO: 26).

In certain embodiments, the polynucleotide sequence encoding a GC-rich sequence comprises CCGGTACCGCGCGCGTCGACGC (SEQ ID NO: 11).

In certain embodiments, the polynucleotide sequence encoding a GC-rich sequence comprises CCGGTACCGCGCGCGTCGA (SEQ ID NO: 12).

In certain embodiments, the polynucleotide sequence encoding a GC-rich sequence comprises CCGGTACCGCGCGCG (SEQ ID NO: 14).

In certain embodiments, the polynucleotide sequence encoding a GC-rich sequence comprises CCGGTACCGCGCGCCTCGAGGC (SEQ ID NO: 16).

In certain embodiments, the polynucleotide sequence encoding a GC-rich sequence comprises CCGGTACCGCGCGCCTCGA (SEQ ID NO: 17).

In certain embodiments, the polynucleotide sequence encoding a GC-rich sequence comprises CCGGTACCGCGCGCC (SEQ ID NO: 19).

In certain embodiments, the polynucleotide sequence encoding a GC-rich sequence comprises CCGGTACCGCGCGCGGATCCGC (SEQ ID NO: 21).

In certain embodiments, the polynucleotide sequence encoding a GC-rich sequence comprises CCGGTACCGCGCGCGGATC (SEQ ID NO: 22).

In certain embodiments, the polynucleotide sequence encoding a GC-rich sequence comprises CCGGTACCGCGCGCG (SEQ ID NO: 24).

In one aspect, the disclosure provides a method for producing a plurality of chemically modified mRNA molecules with similar polyA sequence lengths, comprising the steps of: (a) in vitro transcribing the plurality of mRNA molecules in the presence of at least one chemically modified nucleotide, thereby producing a plurality of chemically modified mRNA molecules; and (b) contacting the chemically modified mRNA molecules with a polyA polymerase under conditions to allow the synthesis of a polyA sequence to the 3′ end of the chemically modified mRNA molecules, thereby producing a plurality of chemically modified mRNA molecules with similar polyA sequence lengths; wherein each mRNA molecule within the plurality of mRNA molecules comprise, from 5′ to 3′, a 5′ untranslated region (5′ UTR), at least one open reading frame (ORF), a 3′ untranslated region (3′ UTR), and a GC-rich sequence.

In certain embodiments, the GC-rich sequence is contained within the 3′ UTR.

In certain embodiments, the GC-rich sequence is not contained within the 3′ UTR.

In certain embodiments, the presence of the GC-rich sequence in each mRNA molecule within the plurality of mRNA molecules facilitates the generation of polyA sequences of substantially the same length.

In certain embodiments, at least 60% of the chemically modified mRNA molecules within the plurality of chemically modified mRNA molecules comprise a polyA sequence length that is substantially the same.

In certain embodiments, about 60%, about 70%, about 80%, about 85% m, about 90%, about 95%, or about 99% of the chemically modified mRNA molecules within the plurality of chemically modified mRNA molecules comprise a polyA sequence length that is substantially the same.

In certain embodiments, substantially all of the chemically modified mRNA molecules within the plurality of chemically modified mRNA molecules comprise a polyA sequence length that is substantially the same.

In certain embodiments, at least 60% of the chemically modified mRNA molecules within the plurality of chemically modified mRNA molecules comprise a polyA sequence length of about 50 to about 100 consecutive adenosine nucleotides, about 100 to about 150 consecutive adenosine nucleotides, about 150 to about 200 consecutive adenosine nucleotides, about 200 to about 250 consecutive adenosine nucleotides, about 250 to about 300 consecutive adenosine nucleotides, about 300 to about 350 consecutive adenosine nucleotides, about 350 to about 400 consecutive adenosine nucleotides, about 400 to about 450 consecutive adenosine nucleotides, or about 450 to about 500 consecutive adenosine nucleotides.

In certain embodiments, about 60%, about 70%, about 80%, about 85% m, about 90%, about 95%, or about 99% of the chemically modified mRNA molecules within the plurality of chemically modified mRNA molecules comprise a polyA sequence length of about 50 to about 100 consecutive adenosine nucleotides, about 100 to about 150 consecutive adenosine nucleotides, about 150 to about 200 consecutive adenosine nucleotides, about 200 to about 250 consecutive adenosine nucleotides, about 250 to about 300 consecutive adenosine nucleotides, about 300 to about 350 consecutive adenosine nucleotides, about 350 to about 400 consecutive adenosine nucleotides, about 400 to about 450 consecutive adenosine nucleotides, or about 450 to about 500 consecutive adenosine nucleotides.

In certain embodiments, substantially all of the chemically modified mRNA molecules within the plurality of chemically modified mRNA molecules comprise a polyA sequence length of about 50 to about 100 consecutive adenosine nucleotides, about 100 to about 150 consecutive adenosine nucleotides, about 150 to about 200 consecutive adenosine nucleotides, about 200 to about 250 consecutive adenosine nucleotides, about 250 to about 300 consecutive adenosine nucleotides, about 300 to about 350 consecutive adenosine nucleotides, about 350 to about 400 consecutive adenosine nucleotides, about 400 to about 450 consecutive adenosine nucleotides, or about 450 to about 500 consecutive adenosine nucleotides.

In certain embodiments, the polyA sequence lengths in the plurality of chemically modified mRNA molecules comprise a polyA sequence length that is within 50%, within 45%, within 40%, within 35%, within 30%, within 25%, within 20%, within 15%, within 10%, or within 5% of a mean polyA sequence length in the plurality of chemically modified mRNA molecules.

In certain embodiments, the polyA sequence length is measured by capillary gel electrophoresis (CGE) or by liquid chromatography (LC).

In certain embodiments, the GC-rich sequence comprises at least about 50% G and/or C nucleotides to 100% G and/or C nucleotides.

In certain embodiments, the GC-rich sequence comprises at least about 70% G and/or C nucleotides.

In certain embodiments, the GC-rich sequence comprises at least about 80% G and/or C nucleotides.

In certain embodiments, the GC-rich sequence comprises at least about 80% G and/or C nucleotides and is at least 14 nucleotides in length.

In certain embodiments, the GC-rich sequence comprises 100% G and/or C nucleotides.

In certain embodiments, the GC-rich sequence comprises CCGGUACCG.

In certain embodiments, the GC-rich sequence comprises

(SEQ ID NO: 1)
CCGGUACCGCGCGC.

In certain embodiments, the GC-rich sequence comprises

(SEQ ID NO: 13)
CCGGUACCGCGCGCGUCGA.

In certain embodiments, the GC-rich sequence comprises

(SEQ ID NO: 15)
CCGGUACCGCGCGC.

In certain embodiments, the GC-rich sequence comprises

(SEQ ID NO: 18)
CCGGUACCGCGCGCCUCGA.

In certain embodiments, the GC-rich sequence comprises

(SEQ ID NO: 20)
CCGGUACCGCGCGCC.

In certain embodiments, the GC-rich sequence comprises

(SEQ ID NO: 23)
CCGGUACCGCGCGCGGAUC.

In certain embodiments, the GC-rich sequence comprises

(SEQ ID NO: 25)
CCGGUACCGCGCGCG.

In certain embodiments, the GC-rich sequence comprises CCG.

In one aspect, the disclosure provides a method for producing a plurality of chemically modified mRNA molecules with polyA sequence lengths of at least about 50 consecutive adenosine nucleotides, comprising the steps of: (a) in vitro transcribing the plurality of mRNA molecules in the presence of at least one chemically modified nucleotide, thereby producing a plurality of chemically modified mRNA molecules; and (b) contacting the chemically modified mRNA molecules with a polyA polymerase under conditions to allow the synthesis of a polyA sequence to the 3′ end of the chemically modified mRNA molecules, thereby producing a plurality of chemically modified mRNA molecules with polyA sequence lengths of at least about 200 consecutive adenosine nucleotides; wherein each mRNA molecule within the plurality of mRNA molecules comprise, from 5′ to 3′, a 5′ untranslated region (5′ UTR), at least one open reading frame (ORF), a 3′ untranslated region (3′ UTR), and a GC-rich sequence.

In certain embodiments, the GC-rich sequence is contained within the 3′ UTR.

In certain embodiments, the GC-rich sequence is not contained within the 3′ UTR.

In certain embodiments, the presence of the GC-rich sequence in each mRNA molecule within the plurality of mRNA molecules facilitates the generation of polyA sequences of substantially the same length of at least about 50 consecutive adenosine nucleotides.

In certain embodiments, at least 60% of the chemically modified mRNA molecules within the plurality of chemically modified mRNA molecules comprise a polyA sequence length that is substantially the same of at least about 50 consecutive adenosine nucleotides.

In certain embodiments, about 60%, about 70%, about 80%, about 85%, about 90%, about 95%, or about 99% of the chemically modified mRNA molecules within the plurality of chemically modified mRNA molecules comprise a polyA sequence length that is substantially the same of at least about 50 consecutive adenosine nucleotides.

In certain embodiments, substantially all of the chemically modified mRNA molecules within the plurality of chemically modified mRNA molecules comprise a polyA sequence length that is substantially the same of at least about 50 consecutive adenosine nucleotides.

In certain embodiments, at least 60% of the chemically modified mRNA molecules within the plurality of chemically modified mRNA molecules comprise a polyA sequence length of about 50 to about 100 consecutive adenosine nucleotides, about 100 to about 150 consecutive adenosine nucleotides, about 150 to about 200 consecutive adenosine nucleotides, about 200 to about 250 consecutive adenosine nucleotides, about 250 to about 300 consecutive adenosine nucleotides, about 300 to about 350 consecutive adenosine nucleotides, about 350 to about 400 consecutive adenosine nucleotides, about 400 to about 450 consecutive adenosine nucleotides, or about 450 to about 500 consecutive adenosine nucleotides.

In certain embodiments, at least 60% of the chemically modified mRNA molecules within the plurality of chemically modified mRNA molecules comprise a polyA sequence length of about 50, about 80, about 100, about 120, about 150, about 180, about 200, about 220, about 250, about 280, about 300, about 320, about 350, about 380, about 400, about 420, about 450, about 480, or about 500 consecutive adenosine nucleotides.

In certain embodiments, about 60%, about 70%, about 80%, about 85%, about 90%, about 95%, or about 99% of the chemically modified mRNA molecules within the plurality of chemically modified mRNA molecules comprise a polyA sequence length of about 50 to about 100 consecutive adenosine nucleotides, about 100 to about 150 consecutive adenosine nucleotides, about 150 to about 200 consecutive adenosine nucleotides, about 200 to about 250 consecutive adenosine nucleotides, about 250 to about 300 consecutive adenosine nucleotides, about 300 to about 350 consecutive adenosine nucleotides, about 350 to about 400 consecutive adenosine nucleotides, about 400 to about 450 consecutive adenosine nucleotides, or about 450 to about 500 consecutive adenosine nucleotides.

In certain embodiments, about 60%, about 70%, about 80%, about 85%, about 90%, about 95%, or about 99% of the chemically modified mRNA molecules within the plurality of chemically modified mRNA molecules comprise a polyA sequence length of about 50, about 80, about 100, about 120, about 150, about 180, about 200, about 220, about 250, about 280, about 300, about 320, about 350, about 380, about 400, about 420, about 450, about 480, or about 500 consecutive adenosine nucleotides.

In certain embodiments, substantially all of the chemically modified mRNA molecules within the plurality of chemically modified mRNA molecules comprise a polyA sequence length of about 50 to about 100 consecutive adenosine nucleotides, about 100 to about 150 consecutive adenosine nucleotides, about 150 to about 200 consecutive adenosine nucleotides, about 200 to about 250 consecutive adenosine nucleotides, about 250 to about 300 consecutive adenosine nucleotides, about 300 to about 350 consecutive adenosine nucleotides, about 350 to about 400 consecutive adenosine nucleotides, about 400 to about 450 consecutive adenosine nucleotides, or about 450 to about 500 consecutive adenosine nucleotides.

In certain embodiments, substantially all of the chemically modified mRNA molecules within the plurality of chemically modified mRNA molecules comprise a polyA sequence length of about 50, about 80, about 100, about 120, about 150, about 180, about 200, about 220, about 250, about 280, about 300, about 320, about 350, about 380, about 400, about 420, about 450, about 480, or about 500 consecutive adenosine nucleotides.

In certain embodiments, the polyA sequence lengths in the plurality of chemically modified mRNA molecules comprise a polyA sequence length that is within 50%, within 45%, within 40%, within 35%, within 30%, within 25%, within 20%, within 15%, within 10%, or within 5% of a mean polyA sequence length in the plurality of chemically modified mRNA molecules.

In certain embodiments, the polyA sequence length is measured by capillary gel electrophoresis (CGE) or by liquid chromatography (LC).

### BRIEF DESCRIPTION OF THE DRAWINGS

FIG. 1 is a schematic of the 3′ end of the 3′ UTR insertion site before and after the GC-rich sequence (i.e., CCGGTACCGCGCGCAAAC, SEQ ID NO: 3) modification. The top strand sequence before modification as depicted in FIG. 1 corresponds to SEQ ID NO: 4 and the bottom strand corresponds to SEQ ID NO: 5. The top strand sequence after modification as depicted in FIG. 1 corresponds to SEQ ID NO: 6 and the bottom strand corresponds to SEQ ID NO: 7.

FIG. 2 depicts a western blot detecting the antigen encoded by the influenza antigen H3/Sing16 expressed by HEK293 cells transfected with the mRNA produced from a BspQI-or BssHII-cut template.

### DETAILED DESCRIPTION OF THE DISCLOSURE

The present disclosure is directed to, inter alia, a messenger RNA (mRNA) comprising, from 5′ to 3′, a 5′ untranslated region (5′ UTR), at least one open reading frame (ORF), a 3′ untranslated region (3′ UTR), and a GC-rich sequence, wherein the mRNA comprises at least one chemical modification. The GC-rich sequence comprises at least about 50% G and/or C nucleotides to 100% G and/or C nucleotides. Enzymatic polyA tailing of chemically modified mRNA has been shown to yield non-uniform polyA tail lengths. It has been surprisingly discovered herein that the placement of a GC-rich sequence at the 3′ end of chemically modified mRNA molecules in a plurality of chemically modified mRNA molecules yields more uniform and sufficiently long polyA tails after an enzymatic polyA tailing reaction with a polyA polymerase.

### I. Definitions

### II.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, virology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, may provide one of skill with a general dictionary of many of the terms used in this disclosure.

Units, prefixes, and symbols are denoted in their International System of Units (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure. Accordingly, the terms defined immediately below are more fully defined by reference to the specification in its entirety.

The term “approximately” or “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some embodiments, the term indicates deviation from the indicated numerical value by ±10%, ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, or ±0.01%. In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%. In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.2%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%.

As used herein, the term “messenger RNA” or “mRNA” refers to a polynucleotide that encodes at least one polypeptide. mRNA may contain one or more coding and non-coding regions. A coding region is alternatively referred to as an open reading frame (ORF). Non-coding regions in mRNA include the 5′ cap, 5′ untranslated region (UTR), 3′ UTR, and a polyA tail. mRNA can be purified from natural sources, produced using recombinant expression systems (e.g., in vitro transcription) and optionally purified, or chemically synthesized.

As used herein, the term “GC-rich sequence” refers to a polynucleotide sequence of at least two nucleotides that is composed of at least 50% G and/or C nucleotides. By way of example, but in no way limiting, the sequences GGAT, GCAT, and CCAT are all GC-rich sequences.

### II. GC-Rich Sequence

The chemically modified mRNA of the disclosure and the DNA templates encoding the same comprise at least one GC-rich sequence at the 3′ end of the mRNA or the 3′ end of the DNA template encoding the same. The GC-rich sequence comprises at least two nucleotides comprising at least 50% G and/or C nucleotides. In certain embodiments, the GC-rich sequence comprises at least about 50% G and/or C nucleotides to 100% G and/or C nucleotides (e.g., at least about 50%, at least about 55%, at least about 60%, at least about 65%, at least about 70%, at least about 75%, at least about 80%, at least about 85%, at least about 90%, at least about 95%, or 100% G and/or C nucleotides). In certain embodiments, the GC-rich sequence comprises at least about 70% G and/or C nucleotides. In certain embodiments, the GC-rich sequence comprises at least about 80% G and/or C nucleotides. In certain embodiments, the GC-rich sequence comprises 100% G and/or C nucleotides.

In certain embodiments, the GC-rich sequence is between 2 and 50 nucleotides in length. In certain embodiments, the rich sequence is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In certain embodiments, the GC-rich sequence is 20 nucleotides in length or less.

In certain embodiments, the GC-rich sequence comprises at least one G nucleotide. In certain embodiments, the GC-rich sequence comprises at least one C nucleotide. In certain embodiments, the GC-rich sequence comprises at least one G nucleotide and at least one C nucleotide.

In certain embodiments, the GC-rich sequence is interrupted by at least one nucleotide different from a guanine or cytosine nucleotide (i.e., an adenine (A) or uracil (U)). In certain embodiments, the GC-rich sequence is interrupted by 1, 2, 3, 4, 5, 6, 7, 8, 9, 10, 11, 12, 13, 14, 15, 16, 17, 18, 19, or 20 nucleotides that are different from a guanine or cytosine nucleotide (i.e., an adenine (A) or uracil (U)).

In certain embodiments, the GC-rich sequence comprises CCGGUACCG. In certain embodiments, the GC-rich sequence comprises CCGGUACCGCGCGC (SEQ ID NO: 1). In certain embodiments, the GC-rich sequence comprises CCG.

The GC-rich sequence of the disclosure is present at the 3′ end of the chemically modified mRNA. In certain embodiments, the GC-rich sequence is contained within the 3′ UTR of the mRNA (i.e., the GC-rich sequence is present at the 3′ end of the 3′ UTR). In other embodiments, the GC-rich sequence is not contained within the 3′ UTR (i.e., the GC-rich sequence is separate and distinct from the 3′ UTR, and is positioned 3′ to the 3′ UTR).

In certain embodiments, the mRNA is expressed in vivo or ex vivo.

In certain embodiments, the mRNA is synthesized using in vitro transcription (IVT).

The GC-rich sequence of the disclosure may be encoded within a DNA polynucleotide used as a template for in vitro transcribing the chemically modified mRNA. In certain embodiments, the disclosure provides a DNA polynucleotide comprising a nucleic acid sequence encoding the mRNA described herein.

In another embodiment, the disclosure provides a vector (i.e., a plasmid) comprising the DNA polynucleotide comprising a nucleic acid sequence encoding the mRNA described herein.

In certain embodiments, the vector comprises at least elements a-c, from 5′ to 3′: a. an RNA polymerase promoter; b. a polynucleotide sequence encoding an ORF; and c. a polynucleotide sequence encoding a GC-rich sequence. In certain embodiments, the vector further comprises: d. a polynucleotide sequence encoding a restriction enzyme recognition site.

In certain embodiments, the vector comprises at least elements a-e, from 5′ to 3′: a. an RNA polymerase promoter; b. a polynucleotide sequence encoding a 5′ UTR; c. a polynucleotide sequence encoding an ORF; d. a polynucleotide sequence encoding a 3′ UTR; and c. a polynucleotide sequence encoding a GC-rich sequence. In certain embodiments, the vector further comprises: f. a polynucleotide sequence encoding a restriction enzyme recognition site. In certain embodiments, the vector further comprises: g. a polynucleotide sequence encoding a polyadenylation signal. In other embodiments, the vector lacks a polynucleotide sequence encoding a polyadenylation signal.

In certain embodiments, the vector comprises at least elements a-d, from 5′ to 3′: a. an RNA polymerase promoter; b. a polynucleotide sequence encoding a 5′ UTR; c. a polynucleotide sequence encoding an ORF; and d. a polynucleotide sequence encoding a 3′ UTR with a GC-rich sequence present at the 3′ end of the 3′UTR. In certain embodiments, the vector further comprises: c. a polynucleotide sequence encoding a restriction enzyme recognition site. In certain embodiments, the vector further comprises: f. a polynucleotide sequence encoding a polyadenylation signal. In other embodiments, the vector lacks a polynucleotide sequence encoding a polyadenylation signal.

In certain embodiments of the vector, the GC-rich sequence comprises CCGGTACCG. In certain embodiments of the vector, the GC-rich sequence comprises CCGGTACCGCGCGC (SEQ ID NO: 2). In certain embodiments of the vector, the GC-rich sequence comprises CCG.

The above recited vectors may be linearized with a restriction enzyme before being used for IVT. As used herein, a “restriction enzyme” is a protein that cleaves DNA sequences at sequence-specific sites (i.e., a “restriction enzyme recognition site”), producing DNA fragments or a linearized DNA vector with a known sequence at each end. The restriction enzyme linearizes the vector such that the linear vector ends with the GC-rich sequence of the disclosure (i.e., the GC-rich sequence is present at the 3′ end of the linearized vector). Any restriction enzyme may be employed in the vectors that yields a 3′ end comprising a GC-rich sequence. In certain embodiments, the restriction enzyme recognition site comprises one or more of a BspQI recognition site, a BssHII recognition site, and a Acc65I recognition site. In certain embodiments, the restriction enzyme recognition site comprises a BspQI recognition site. In certain embodiments, the vector is linearized with a BspQI recognition site. In certain embodiments, the restriction enzyme recognition site comprises a BssHII recognition site. In certain embodiments, the vector is linearized with a BssHII recognition site. In certain embodiments, the restriction enzyme recognition site comprises a Acc65I recognition site. In certain embodiments, the vector is linearized with a Acc65I recognition site.

### III. RNA

The present compositions of the disclosure comprise a chemically modified RNA molecule (e.g., mRNA) that encodes a polypeptide (e.g., an antigenic polypeptide). The RNA molecule of the present disclosure comprises at least one ribonucleic acid (RNA) comprising an ORF encoding a polypeptide. In certain embodiments, the RNA is a messenger RNA (mRNA) comprising an ORF encoding a polypeptide.

In certain embodiments, the polypeptide is a biologically active polypeptide, a therapeutic polypeptide, or an antigenic polypeptide.

In certain embodiments, the antigenic polypeptide is derived from a pathogen. In certain embodiments, the pathogen is a viral pathogen or prokaryotic pathogen. When the mRNA of the disclosure encodes an antigenic polypeptide, the mRNA may be administered to a subject as a vaccine.

In certain embodiments, the polypeptide comprises an antibody or fragment thereof, enzyme replacement polypeptide, or genome-editing polypeptide.

In certain embodiments, the therapeutic polypeptide comprises an antibody heavy chain, an antibody light chain, an enzyme, or a cytokine.

In certain embodiments, the biologically active polypeptide comprises a genome-editing polypeptide (e.g., an RNA-guide nuclease, a zinc finger nuclease, a TALEN, or a meganuclease).

In certain embodiments, the RNA (e.g., mRNA) further comprises at least one 5′ UTR, 3′ UTR, a poly(A) tail, and/or a 5′ cap.

### A. 5′ Cap

An mRNA 5′ cap can provide resistance to nucleases found in most eukaryotic cells and promote translation efficiency. Several types of 5′ caps are known. A 7-methylguanosine cap (also referred to as “m⁷G” or “Cap-0”), comprises a guanosine that is linked through a 5′-5′-triphosphate bond to the first transcribed nucleotide.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp, (5′(A,G(5′)ppp(5′)A, and G(5′)ppp(5′)G. Additional cap structures are described in U.S. Publication No. US 2016/0032356 and U.S. Publication No. US 2018/0125989, which are incorporated herein by reference.

5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′)G (the ARCA cap); G(5′)ppp(5′)A; G(5′)ppp(5′)G; m7G(5′)ppp(5′)A; m7G(5′)ppp(5′)G; m7G(5′)ppp(5′)(2′OMeA)pG; m7G(5′)ppp(5′)(2′OMeA)pU; m7G(5′)ppp(5′)(2′OMeG)pG (New England BioLabs, Ipswich, MA; TriLink Biotechnologies). 5′-capping of modified RNA may be completed post-transcriptionally using a vaccinia virus capping enzyme to generate the Cap 0 structure: m7G(5′)ppp(5′)G. Cap 1 structure may be generated using both vaccinia virus capping enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase.

In certain embodiments, the mRNA of the disclosure comprises a 5′ cap selected from the group consisting of 3′-O-Me-m7G(5′)ppp(5′)G (the ARCA cap), G(5′)ppp(5′)A, G(5′)ppp(5′)G, m7G(5′)ppp(5′)A, m7G(5′)ppp(5′)G, m7G(5′)ppp(5′)(2′OMeA)pG, m7G(5′)ppp(5′)(2′OMeA)pU, and m7G(5′)ppp(5′)(2′OMeG)pG.

In certain embodiments, the mRNA of the disclosure comprises a 5′ cap of:

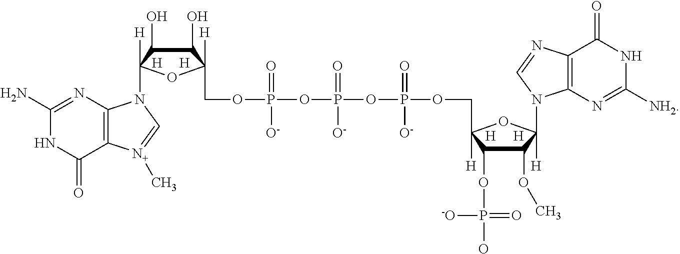

### B. Untranslated Region (UTR)

In some embodiments, the mRNA of the disclosure includes a 5′ and/or 3′ untranslated region (UTR). In mRNA, the 5′ UTR starts at the transcription start site and continues to the start codon but does not include the start codon. The 3′ UTR starts immediately following the stop codon and continues until the transcriptional termination signal.

In some embodiments, the mRNA disclosed herein may comprise a 5′ UTR that includes one or more elements that affect an mRNA's stability or translation. In some embodiments, a 5′ UTR may be about 10 to 5,000 nucleotides in length. In some embodiments, a 5′ UTR may be about 50 to 500 nucleotides in length. In some embodiments, the 5′ UTR is at least about 10 nucleotides in length, about 20 nucleotides in length, about 30 nucleotides in length, about 40 nucleotides in length, about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length or about 5,000 nucleotides in length.

In some embodiments, the mRNA disclosed herein may comprise a 3′ UTR comprising one or more of a polyadenylation signal, a binding site for proteins that affect an mRNA's stability of location in a cell, or one or more binding sites for miRNAs. In some embodiments, a 3′ UTR may be 50 to 5,000 nucleotides in length or longer. In some embodiments, a 3′ UTR may be 50 to 1,000 nucleotides in length or longer. In some embodiments, the 3′ UTR is at least about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length, or about 5,000 nucleotides in length.

In certain embodiments, the 3′ UTR comprises the GC-rich sequence described herein. In certain embodiments, the GC-rich sequence is present at the 3′ end of the 3′ UTR. In other embodiments, the 3′ UTR does not comprise the GC-rich sequence.

In some embodiments, the mRNA disclosed herein may comprise a 5′ or 3′ UTR that is derived from a gene distinct from the one encoded by the mRNA transcript (i.e., the UTR is a heterologous UTR).

In certain embodiments, the 5′ and/or 3′ UTR sequences can be derived from mRNA which are stable (e.g., globin, actin, GAPDH, tubulin, histone, or citric acid cycle enzymes) to increase the stability of the mRNA. For example, a 5′ UTR sequence may include a partial sequence of a CMV immediate-early 1 (IE1) gene, or a fragment thereof, to improve the nuclease resistance and/or improve the half-life of the mRNA. Also contemplated is the inclusion of a sequence encoding human growth hormone (hGH), or a fragment thereof, to the 3′ end or untranslated region of the mRNA. Generally, these modifications improve the stability and/or pharmacokinetic properties (e.g., half-life) of the mRNA relative to their unmodified counterparts, and include, for example, modifications made to improve such mRNA resistance to in vivo nuclease digestion.

Exemplary 5′ UTRs include a sequence derived from a CMV immediate-early 1 (IE1) gene (U.S. Publication Nos. 2014/0206753 and 2015/0157565, each of which is incorporated herein by reference), or the sequence GGGAUCCUACC (SEQ ID NO: 8) (U.S. Publication No. 2016/0151409, incorporated herein by reference).

In various embodiments, the 5′ UTR may be derived from the 5′ UTR of a TOP gene. TOP genes are typically characterized by the presence of a 5′-terminal oligopyrimidine (TOP) tract. Furthermore, most TOP genes are characterized by growth-associated translational regulation. However, TOP genes with a tissue specific translational regulation are also known. In certain embodiments, the 5′ UTR derived from the 5′ UTR of a TOP gene lacks the 5′ TOP motif (the oligopyrimidine tract) (e.g., U.S. Publication Nos. 2017/0029847, 2016/0304883, 2016/0235864, and 2016/0166710, each of which is incorporated herein by reference).

In certain embodiments, the 5′ UTR is derived from a ribosomal protein Large 32 (L32) gene (U.S. Publication No. 2017/0029847, supra).

In certain embodiments, the 5′ UTR is derived from the 5′ UTR of an hydroxysteroid (17-b) dehydrogenase 4 gene (HSD17B4) (U.S. Publication No. 2016/0166710, supra).

In certain embodiments, the 5′ UTR is derived from the 5′ UTR of an ATP5A1 gene (U.S. Publication No. 2016/0166710, supra).

In some embodiments, an internal ribosome entry site (IRES) is used instead of a 5′ UTR.

In some embodiments, the 5′UTR comprises a nucleic acid sequence set forth in SEQ ID NO: 9 and reproduced below:

(SEQ ID NO: 9)
GGACAGAUCGCCUGGAGACGCCAUCCACGCUGUUUUGACCUCCAUAGAA
GACACCGGGACCGAUCCAGCCUCCGCGGCCGGGAACGGUGCAUUGGAAC
GCGGAUUCCCCGUGCCAAGAGUGACUCACCGUCCUUGACACG.

In some embodiments, the 3′UTR comprises a nucleic acid sequence set forth in SEQ ID NO: 10 and reproduced below:

(SEQ ID NO: 10)
CGGGUGGCAUCCCUGUGACCCCUCCCCAGUGCCUCUCCUGGCCCUGGAA
GUUGCCACUCCAGUGCCCACCAGCCUUGUCCUAAUAAAAUUAAGUUGCA
UC.

The 5′ UTR and 3′UTR are described in further detail in WO2012/075040, incorporated herein by reference.

### C. Polyadenylated Tail

As used herein, the terms “polyA sequence,” “polyA tail,” and “polyA region” refer to a sequence of adenosine nucleotides at the 3′ end of the mRNA molecule. The chemically modified mRNA of the disclosure may further comprise a polyA tail. The polyA tail may confer stability to the mRNA and protect it from exonuclease degradation. The polyA tail may enhance translation. In some embodiments, the polyA tail is essentially homopolymeric. For example, a polyA tail of 100 adenosine nucleotides may have essentially a length of 100 nucleotides.

The “polyA tail,” as used herein, typically relates to RNA. However, in the context of the disclosure, the term likewise relates to corresponding sequences in a DNA molecule (e.g., a “polyT sequence”).

The polyA tail may comprise about 10 to about 500 adenosine nucleotides, about 10 to about 300 adenosine nucleotides, about 40 to about 300 adenosine nucleotides, about 80 to about 300, about 10 to about 200, about 40 to about 200, or about 40 to about 150 adenosine nucleotides. The length of the polyA tail may be at least about 10, 20, 30, 40, 50, 75, 100, 150, 200, 250, 300, 350, 400, 450, or 500 adenosine nucleotides. In certain embodiments, the adenosine nucleotides are consecutive.

In some embodiments where the nucleic acid is an RNA, the polyA tail of the nucleic acid is obtained from a DNA template during RNA in vitro transcription. In certain embodiments, the polyA tail is obtained in vitro by common methods of chemical synthesis without being transcribed from a DNA template. In various embodiments, polyA tails are generated by enzymatic polyadenylation of the RNA (after RNA in vitro transcription) using commercially available polyadenylation kits and corresponding protocols, or alternatively, by using immobilized polyA polymerases, e.g., using methods and means as described in WO2016/174271.

The nucleic acid may comprise a polyA tail obtained by enzymatic polyadenylation, wherein the majority of nucleic acid molecules comprise about 100 (+/−20) to about 500 (+/−50) or about 250 (+/−20) adenosine nucleotides.

In some embodiments, the nucleic acid may comprise a polyA tail derived from a template DNA and may additionally comprise at least one additional polyA tail generated by enzymatic polyadenylation, e.g., as described in WO2016/091391.

In certain embodiments, the nucleic acid comprises at least one polyadenylation signal.

### D. Chemical Modification

The mRNA disclosed herein comprise at least one chemical modification. In some embodiments, the mRNA disclosed herein may contain one or more modifications that typically enhance RNA stability. Exemplary modifications can include backbone modifications, sugar modifications, or base modifications. In some embodiments, the disclosed mRNA may be synthesized from naturally occurring nucleotides and/or nucleotide analogues (modified nucleotides) including, but not limited to, purines (adenine (A) and guanine (G)) or pyrimidines (thymine (T), cytosine (C), and uracil (U)). In certain embodiments, the disclosed mRNA may be synthesized from modified nucleotide analogues or derivatives of purines and pyrimidines, such as, e.g., 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxy acetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, β-D-mannosyl-queosine, phosphoramidates, phosphorothioates, peptide nucleotides, methylphosphonates, 7-deazaguanosine, 5-methylcytosine, and inosine.

In some embodiments, the disclosed mRNA may comprise at least one chemical modification including, but not limited to, pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

In some embodiments, the chemical modification is selected from the group consisting of pseudouridine, N1-methylpseudouridine, 5-methylcytosine, 5-methoxyuridine, and a combination thereof.

In some embodiments, the chemical modification comprises N1-methylpseudouridine.

In some embodiments, at least 20%, at least 30%, at least 40%, at least 50%, at least 60%, at least 70%, at least 80%, at least 85%, at least 90%, at least 95%, or 100% of the uracil nucleotides in the mRNA are chemically modified.

In some embodiments, at least 20%, at least 30%, at least 40%, at least 50%, at least 60%, at least 70%, at least 80%, at least 85%, at least 90%, at least 95%, or 100% of the uracil nucleotides in the ORF are chemically modified.

The preparation of such analogues is described, e.g., in U.S. Pat. Nos. 4,373,071, 4,401,796, 4,415,732, 4,458,066, 4,500,707, 4,668,777, 4,973,679, 5,047,524, 5,132,418, 5,153,319, 5,262,530, and 5,700,642.

E. mRNA Synthesis

The mRNAs disclosed herein may be synthesized according to any of a variety of methods. For example, mRNAs according to the present disclosure may be synthesized via in vitro transcription (IVT). Some methods for in vitro transcription are described, e.g., in Geall et al. (2013) Semin. Immunol. 25(2): 152-159; Brunelle et al. (2013) Methods Enzymol. 530:101-14. Briefly, IVT is typically performed with a linear or circular DNA template containing a promoter, a pool of ribonucleotide triphosphates, a buffer system that may include DTT and magnesium ions, an appropriate RNA polymerase (e.g., T3, T7, or SP6 RNA polymerase), DNase I, pyrophosphatase, and/or RNase inhibitor. The exact conditions may vary according to the specific application. The presence of these reagents is generally undesirable in a final mRNA product and these reagents can be considered impurities or contaminants which can be purified or removed to provide a clean and/or homogeneous mRNA that is suitable for therapeutic use. While mRNA provided from in vitro transcription reactions may be desirable in some embodiments, other sources of mRNA can be used according to the instant disclosure including wild-type mRNA produced from bacteria, fungi, plants, and/or animals.

### IV. Vectors

In one aspect, disclosed herein are vectors comprising the mRNA compositions disclosed herein. The RNA sequences encoding a protein of interest (e.g., mRNA encoding an antigenic prokaryotic polypeptide) can be cloned into a number of types of vectors. For example, the nucleic acids can be cloned into a vector including, but not limited to, a plasmid, a phagemid, a phage derivative, an animal virus, and a cosmid. Vectors of particular interest can include expression vectors, replication vectors, probe generation vectors, sequencing vectors, and vectors optimized for in vitro transcription.

In certain embodiments, the vector can be used to express mRNA in a host cell. In various embodiments, the vector can be used as a template for IVT. The construction of optimally translated IVT mRNA suitable for therapeutic use is disclosed in detail in Sahin, et al. (2014). Nat. Rev. Drug Discov. 13, 759-780; Weissman (2015). Expert Rev. Vaccines 14, 265-281.

In certain embodiments, the vector comprises at least elements a-c, from 5′ to 3′: a. an RNA polymerase promoter; b. a polynucleotide sequence encoding an ORF; and c. a polynucleotide sequence encoding a GC-rich sequence. In certain embodiments, the vector further comprises: d. a polynucleotide sequence encoding a restriction enzyme recognition site.

In certain embodiments, the vector comprises at least elements a-e, from 5′ to 3′: a. an RNA polymerase promoter; b. a polynucleotide sequence encoding a 5′ UTR; c. a polynucleotide sequence encoding an ORF; d. a polynucleotide sequence encoding a 3′ UTR; and e. a polynucleotide sequence encoding a GC-rich sequence. In certain embodiments, the vector further comprises: f. a polynucleotide sequence encoding a restriction enzyme recognition site. In certain embodiments, the vector further comprises: g. a polynucleotide sequence encoding a polyadenylation signal. In other embodiments, the vector lacks a polynucleotide sequence encoding a polyadenylation signal.

In certain embodiments, the vector comprises at least elements a-d, from 5′ to 3′: a. an RNA polymerase promoter; b. a polynucleotide sequence encoding a 5′ UTR; c. a polynucleotide sequence encoding an ORF; and d. a polynucleotide sequence encoding a 3′ UTR with a GC-rich sequence present at the 3′ end of the 3′UTR. In certain embodiments, the vector further comprises: c. a polynucleotide sequence encoding a restriction enzyme recognition site. In certain embodiments, the vector further comprises: f. a polynucleotide sequence encoding a polyadenylation signal. In other embodiments, the vector lacks a polynucleotide sequence encoding a polyadenylation signal.

A variety of RNA polymerase promoters are known. In some embodiments, the promoter can be a T7 RNA polymerase promoter. Other useful promoters can include, but are not limited to, T3 and SP6 RNA polymerase promoters. Consensus nucleotide sequences for T7, T3, and SP6 promoters are known.

Also disclosed herein are host cells (e.g., mammalian cells, e.g., human cells) comprising the vectors or RNA compositions disclosed herein.

Polynucleotides can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendorf, Hamburg, Germany), cationic liposome mediated transfection using lipofection, polymer encapsulation, peptide mediated transfection, biolistic particle delivery systems such as “gene guns” (see, for example, Nishikawa, et al. (2001). Hum Gene Ther. 12(8):861-70, or the TransIT-RNA transfection Kit (Mirus, Madison, WI).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Regardless of the method used to introduce exogenous nucleic acids into a host cell or otherwise expose a cell to the inhibitor of the present disclosure, in order to confirm the presence of the mRNA sequence in the host cell a variety of assays may be performed.

### V. Pharmaceutical Compositions

The mRNA described herein can be useful as a component in pharmaceutical compositions, for example, for use as a vaccine. These compositions will typically include mRNA and a pharmaceutically acceptable carrier. A pharmaceutical composition of the present disclosure can also include one or more additional components such as small molecule immunopotentiators (e.g., TLR agonists). A pharmaceutical composition of the present disclosure can also include a delivery system for the mRNA, such as a liposome, an oil-in-water emulsion, or a microparticle. In some embodiments, the pharmaceutical composition comprises a lipid nanoparticle (LNP). In certain embodiments, the composition comprises an mRNA comprising at least one chemical modification and a GC-rich sequence at the 3′ end, encapsulated within an LNP.

### VI. Methods for Producing mRNA

In one aspect, the disclosure provides a method for producing a plurality of chemically modified mRNA molecules with similar polyA sequence lengths, comprising the steps of: (a) in vitro transcribing the plurality of mRNA molecules in the presence of at least one chemically modified nucleotide, thereby producing a plurality of chemically modified mRNA molecules; and (b) contacting the chemically modified mRNA molecules with a polyA polymerase under conditions to allow the synthesis of a polyA sequence to the 3′ end of the chemically modified mRNA molecules, thereby producing a plurality of chemically modified mRNA molecules with similar polyA sequence lengths; wherein each mRNA molecule within the plurality of mRNA molecules comprise, from 5′ to 3′, a 5′ untranslated region (5′ UTR), at least one open reading frame (ORF), a 3′ untranslated region (3′ UTR), and a GC-rich sequence.

As used herein, the terms “similar polyA sequence lengths” or “a polyA sequence length that is substantially the same” refer to a plurality of polyA sequence lengths where the polyA sequences in the plurality comprise a polyA sequence length that is within 50% of the mean polyA sequence length. The mean polyA sequence length in a sample with a plurality of polyA sequence lengths is readily determined using a variety of techniques in the art, including but not limited to, capillary gel electrophoresis (CGE) or agarose gel electrophoresis, liquid chromatography (LC), polyA test (PAT) assays, and next-generation sequencing assays, such as TAIL-seq and PAL-seq. These polyA tail length determination methods are described in further detail in Joachimiak et al. (Cells. 11: 677. 2022), incorporated herein by reference.

In certain embodiments, the polyA sequences in the plurality comprise a polyA sequence length that is within 50%, within 45%, within 40%, within 35%, within 30%, within 25%, within 20%, within 15%, within 10%, or within 5% of the mean polyA sequence length in the plurality.

In certain embodiments, at least 70% of the polyA sequences in the plurality comprise a polyA sequence length that is within 50 adenosine nucleotides of each other for polyA tail lengths of 150 adenosine nucleotides or greater. In certain embodiments, at least 75%, at least 80%, at least 90%, at least 95%, or at least 99% of the polyA sequences in the plurality comprise a polyA sequence length that is within 50 adenosine nucleotides of each other for polyA tail lengths of 150 adenosine nucleotides or greater.

In certain embodiments, the GC-rich sequence is contained within the 3′ UTR. In certain embodiments, the GC-rich sequence is not contained within the 3′ UTR.

In certain embodiments, the presence of the GC-rich sequence in each mRNA molecule within the plurality of mRNA molecules facilitates the generation of polyA sequences of similar polyA sequence lengths.

In certain embodiments, at least 60% of the chemically modified mRNA molecules within the plurality of chemically modified mRNA molecules comprise a polyA sequence length that is substantially the same.

In certain embodiments, about 60%, about 70%, about 80%, about 85%, about 90%, about 95%, or about 99% of the chemically modified mRNA molecules within the plurality of chemically modified mRNA molecules comprise a polyA sequence length that is substantially the same.

In certain embodiments, substantially all of the chemically modified mRNA molecules within the plurality of chemically modified mRNA molecules comprise a polyA sequence length that is substantially the same.

In certain embodiments, at least 60% of the chemically modified mRNA molecules within the plurality of chemically modified mRNA molecules comprise a polyA sequence length of about 50 to about 100 consecutive adenosine nucleotides, about 100 to about 150 consecutive adenosine nucleotides, about 150 to about 200 consecutive adenosine nucleotides, about 200 to about 250 consecutive adenosine nucleotides, about 250 to about 300 consecutive adenosine nucleotides, about 300 to about 350 consecutive adenosine nucleotides, about 350 to about 400 consecutive adenosine nucleotides, about 400 to about 450 consecutive adenosine nucleotides, or about 450 to about 500 consecutive adenosine nucleotides.

In certain embodiments, about 60%, about 70%, about 80%, about 85% m, about 90%, about 95%, or about 99% of the chemically modified mRNA molecules within the plurality of chemically modified mRNA molecules comprise a polyA sequence length of about 50 to about 100 consecutive adenosine nucleotides, about 100 to about 150 consecutive adenosine nucleotides, about 150 to about 200 consecutive adenosine nucleotides, about 200 to about 250 consecutive adenosine nucleotides, about 250 to about 300 consecutive adenosine nucleotides, about 300 to about 350 consecutive adenosine nucleotides, about 350 to about 400 consecutive adenosine nucleotides, about 400 to about 450 consecutive adenosine nucleotides, or about 450 to about 500 consecutive adenosine nucleotides.

In certain embodiments, substantially all of the chemically modified mRNA molecules within the plurality of chemically modified mRNA molecules comprise a polyA sequence length of about 50 to about 100 consecutive adenosine nucleotides, about 100 to about 150 consecutive adenosine nucleotides, about 150 to about 200 consecutive adenosine nucleotides, about 200 to about 250 consecutive adenosine nucleotides, about 250 to about 300 consecutive adenosine nucleotides, about 300 to about 350 consecutive adenosine nucleotides, about 350 to about 400 consecutive adenosine nucleotides, about 400 to about 450 consecutive adenosine nucleotides, or about 450 to about 500 consecutive adenosine nucleotides.

In certain embodiments, the polyA sequence length is measured by capillary gel electrophoresis (CGE) or by liquid chromatography (LC).

In another aspect, the disclosure provides a method for producing a plurality of chemically modified mRNA molecules with polyA sequence lengths of at least about 200 consecutive adenosine nucleotides, comprising the steps of: (a) in vitro transcribing the plurality of mRNA molecules in the presence of at least one chemically modified nucleotide, thereby producing a plurality of chemically modified mRNA molecules; and (b) contacting the chemically modified mRNA molecules with a polyA polymerase under conditions to allow the synthesis of a polyA sequence to the 3′ end of the chemically modified mRNA molecules, thereby producing a plurality of chemically modified mRNA molecules with polyA sequence lengths of at least about 200 consecutive adenosine nucleotides; wherein each mRNA molecule within the plurality of mRNA molecules comprise, from 5′ to 3′, a 5′ untranslated region (5′ UTR), at least one open reading frame (ORF), a 3′ untranslated region (3′ UTR), and a GC-rich sequence.

The present invention comprises the following embodiments.

Embodiment 1. A messenger RNA (mRNA) comprising, from 5′ to 3′, a 5′ untranslated region (5′ UTR), at least one open reading frame (ORF), a 3′ untranslated region (3′ UTR), and a GC-rich sequence, wherein the mRNA comprises at least one chemical modification.

Embodiment 2. The mRNA of Embodiment 1, wherein the GC-rich sequence comprises at least about 50% G and/or C nucleotides to 100% G and/or C nucleotides.

Embodiment 3. The mRNA of Embodiment 1, wherein the GC-rich sequence comprises at least about 70% G and/or C nucleotides.

Embodiment 4. The mRNA of Embodiment 1, wherein the GC-rich sequence comprises at least about 80% G and/or C nucleotides.

Embodiment 5. The mRNA of Embodiment 1, wherein the GC-rich sequence comprises 100% G and/or C nucleotides.

Embodiment 6. The mRNA of any one of Embodiments 1-3, wherein the GC-rich sequence comprises CCGGUACCG.

Embodiment 7. The mRNA of any one of Embodiments 1-4, wherein the GC-rich sequence comprises CCGGUACCGCGCGC (SEQ ID NO: 1).

Embodiment 8. The mRNA of any one of Embodiments 1-4, wherein the GC-rich sequence comprises CCGGUACCGCGCGCGUCGA (SEQ ID NO: 13).

Embodiment 9. The mRNA of any one of Embodiments 1-4, wherein the GC-rich sequence comprises CCGGUACCGCGCGC (SEQ ID NO: 15).

Embodiment 10. The mRNA of any one of Embodiments 1-4, wherein the GC-rich sequence comprises CCGGUACCGCGCGCCUCGA (SEQ ID NO: 18).

Embodiment 11. The mRNA of any one of Embodiments 1-4, wherein the GC-rich sequence comprises CCGGUACCGCGCGCC (SEQ ID NO: 20).

Embodiment 12. The mRNA of any one of Embodiments 1-4, wherein the GC-rich sequence comprises CCGGUACCGCGCGCGGAUC (SEQ ID NO: 23).

Embodiment 13. The mRNA of any one of Embodiments 1-4, wherein the GC-rich sequence comprises CCGGUACCGCGCGCG (SEQ ID NO: 25).

Embodiment 14. The mRNA of any one of Embodiments 1-5, wherein the GC-rich sequence comprises CCG.

Embodiment 15. The mRNA of any one of Embodiments 1-14, wherein the GC-rich sequence is contained within the 3′ UTR.

Embodiment 16. The mRNA of any one of Embodiments 1-14, wherein the GC-rich sequence is not contained within the 3′ UTR.

Embodiment 17. The mRNA of any one of Embodiments 1-16, wherein the chemical modification is pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, or 2′-O-methyl uridine.

Embodiment 18. The mRNA of any one of Embodiments 1-16, wherein the chemical modification is pseudouridine, N1-methylpseudouridine, 5-methylcytosine, 5-methoxyuridine, or a combination thereof.

Embodiment 19. The mRNA of any one of Embodiments 1-16, wherein the chemical modification is N1-methylpseudouridine.

Embodiment 20. The mRNA of any one of Embodiments 1-19, further comprising a polyA sequence.

Embodiment 21. The mRNA of Embodiment 20, wherein the polyA sequence is present in the mRNA without enzymatic addition.

Embodiment 22. The mRNA of Embodiment 20 or 21, wherein the polyA sequence is at least 10 consecutive adenosine nucleotides.

Embodiment 23. The mRNA of any one of Embodiments 20-22, wherein the polyA sequence is between 10 and 500 consecutive adenosine nucleotides.

Embodiment 24. The mRNA of any one of Embodiments 20-22, wherein the polyA sequence is between 80 and 300 consecutive adenosine nucleotides.

Embodiment 25. The mRNA of any one of Embodiments 1-24, wherein the mRNA contains a chimeric 5′ or 3′ UTR.

Embodiment 26. The mRNA of any one of Embodiments 1-25, wherein the mRNA encodes at least one polypeptide.

Embodiment 27. The mRNA of Embodiment 26, wherein the polypeptide is a biologically active polypeptide, a therapeutic polypeptide, or an antigenic polypeptide.

Embodiment 28. The mRNA of Embodiment 27, wherein the antigenic polypeptide is derived from a pathogen.

Embodiment 29. The mRNA of Embodiment 28, wherein the polypeptide comprises an antibody or fragment thereof, enzyme replacement polypeptide, or genome-editing polypeptide.

Embodiment 30. The mRNA of Embodiment 29, wherein the therapeutic polypeptide comprises an antibody heavy chain, an antibody light chain, an enzyme, or a cytokine.

Embodiment 31. The mRNA of Embodiment 29, wherein the biologically active polypeptide comprises a genome-editing polypeptide.

Embodiment 32. The mRNA of any one of Embodiments 1-31, wherein the mRNA is synthesized using in vitro transcription (IVT).

Embodiment 33. The mRNA of any one of Embodiments 1-31, wherein the mRNA is expressed in vivo or ex vivo.

Embodiment 34. A DNA polynucleotide comprising a nucleic acid sequence encoding the mRNA of any one of Embodiments 1-33.

Embodiment 35. A vector comprising the DNA polynucleotide of Embodiment 34.

Embodiment 36. The vector of Embodiment 35, wherein the vector comprises at least elements a-c, from 5′ to 3′:
a. an RNA polymerase promoter;
b. a polynucleotide sequence encoding an ORF; and
c. a polynucleotide sequence encoding a GC-rich sequence.

Embodiment 37. The vector of Embodiment 36, further comprising:
d. a polynucleotide sequence encoding a restriction enzyme recognition site.

Embodiment 38. The vector of Embodiment 35, wherein the vector comprises at least elements a-e, from 5′ to 3′:
a. an RNA polymerase promoter;
b. a polynucleotide sequence encoding a 5′ UTR;
c. a polynucleotide sequence encoding an ORF;
d. a polynucleotide sequence encoding a 3′ UTR; and
e. a polynucleotide sequence encoding a GC-rich sequence.

Embodiment 39. The vector of Embodiment 38, further comprising:
f. a polynucleotide sequence encoding a restriction enzyme recognition site.

Embodiment 40. The vector of Embodiment 38 or 39, further comprising:
g. a polynucleotide sequence encoding a polyadenylation signal.

Embodiment 41. The vector of any one of Embodiments 35-40, wherein the vector lacks a polynucleotide sequence encoding a polyadenylation signal.

Embodiment 42. The vector of Embodiment 35, wherein the vector comprises at least elements a-d, from 5′ to 3′:
a. an RNA polymerase promoter;
b. a polynucleotide sequence encoding a 5′ UTR;
c. a polynucleotide sequence encoding an ORF; and
d. a polynucleotide sequence encoding a 3′ UTR with a GC-rich sequence present at the 3′ end of the 3′UTR.

Embodiment 43. The vector of Embodiment 42, further comprising:
e. a polynucleotide sequence encoding a restriction enzyme recognition site.

Embodiment 44. The vector of Embodiment 42 or 43, further comprising:
f. a polynucleotide sequence encoding a polyadenylation signal.

Embodiment 45. The vector of Embodiment 42 or 43, wherein the vector lacks a polynucleotide sequence encoding a polyadenylation signal.

Embodiment 46. The vector of any one of Embodiments 35-45, wherein the restriction enzyme recognition site comprises one or more of a BspQI recognition site, a BssHII recognition site, a SalI recognition site, a XhoI recognition site, a BamHI recognition site, and a Acc65I recognition site.

Embodiment 47. A host cell comprising the vector of Embodiments 35-46.

Embodiment 48. A pharmaceutical composition comprising the mRNA of any one of Embodiments 1-33.

Embodiment 49. A method for producing a plurality of chemically modified mRNA molecules with similar polyA sequence lengths, comprising the steps of:
(a) in vitro transcribing the plurality of mRNA molecules in the presence of at least one chemically modified nucleotide, thereby producing a plurality of chemically modified mRNA molecules; and
(b) contacting the chemically modified mRNA molecules with a polyA polymerase under conditions to allow the synthesis of a polyA sequence to the 3′ end of the chemically modified mRNA molecules, thereby producing a plurality of chemically modified mRNA molecules with similar polyA sequence lengths;
wherein each mRNA molecule within the plurality of mRNA molecules comprise, from 5′ to 3′, a 5′ untranslated region (5′ UTR), at least one open reading frame (ORF), a 3′ untranslated region (3′ UTR), and a GC-rich sequence.

Embodiment 50. The method of Embodiment 49, wherein the GC-rich sequence is contained within the 3′ UTR.

Embodiment 51. The method of Embodiment 49, wherein the GC-rich sequence is not contained within the 3′ UTR.

Embodiment 52. The method of any one of Embodiments 49-51, wherein the presence of the GC-rich sequence in each mRNA molecule within the plurality of mRNA molecules facilitates the generation of polyA sequences of substantially the same length.

Embodiment 53. The method of any one of Embodiments 49-52, wherein at least 60% of the chemically modified mRNA molecules within the plurality of chemically modified mRNA molecules comprise a polyA sequence length that is substantially the same.

Embodiment 54. The method of any one of Embodiments 49-53, wherein about 60%, about 70%, about 80%, about 85% m, about 90%, about 95%, or about 99% of the chemically modified mRNA molecules within the plurality of chemically modified mRNA molecules comprise a polyA sequence length that is substantially the same.

Embodiment 55. The method of any one of Embodiments 49-54, wherein substantially all of the chemically modified mRNA molecules within the plurality of chemically modified mRNA molecules comprise a polyA sequence length that is substantially the same.

Embodiment 56. The method of any one of Embodiments 49-55, wherein at least 60% of the chemically modified mRNA molecules within the plurality of chemically modified mRNA molecules comprise a polyA sequence length of about 50 to about 100 consecutive adenosine nucleotides, about 100 to about 150 consecutive adenosine nucleotides, about 150 to about 200 consecutive adenosine nucleotides, about 200 to about 250 consecutive adenosine nucleotides, about 250 to about 300 consecutive adenosine nucleotides, about 300 to about 350 consecutive adenosine nucleotides, about 350 to about 400 consecutive adenosine nucleotides, about 400 to about 450 consecutive adenosine nucleotides, or about 450 to about 500 consecutive adenosine nucleotides.

Embodiment 57. The method of any one of Embodiments 49-56, wherein about 60%, about 70%, about 80%, about 85% m, about 90%, about 95%, or about 99% of the chemically modified mRNA molecules within the plurality of chemically modified mRNA molecules comprise a polyA sequence length of about 50 to about 100 consecutive adenosine nucleotides, about 100 to about 150 consecutive adenosine nucleotides, about 150 to about 200 consecutive adenosine nucleotides, about 200 to about 250 consecutive adenosine nucleotides, about 250 to about 300 consecutive adenosine nucleotides, about 300 to about 350 consecutive adenosine nucleotides, about 350 to about 400 consecutive adenosine nucleotides, about 400 to about 450 consecutive adenosine nucleotides, or about 450 to about 500 consecutive adenosine nucleotides.

Embodiment 58. The method of any one of Embodiments 49-57, wherein substantially all of the chemically modified mRNA molecules within the plurality of chemically modified mRNA molecules comprise a polyA sequence length of about 50 to about 100 consecutive adenosine nucleotides, about 100 to about 150 consecutive adenosine nucleotides, about 150 to about 200 consecutive adenosine nucleotides, about 200 to about 250 consecutive adenosine nucleotides, about 250 to about 300 consecutive adenosine nucleotides, about 300 to about 350 consecutive adenosine nucleotides, about 350 to about 400 consecutive adenosine nucleotides, about 400 to about 450 consecutive adenosine nucleotides, or about 450 to about 500 consecutive adenosine nucleotides.

Embodiment 59. The method of any one of Embodiments 49-58, wherein the polyA sequence lengths in the plurality of chemically modified mRNA molecules comprise a polyA sequence length that is within 50%, within 45%, within 40%, within 35%, within 30%, within 25%, within 20%, within 15%, within 10%, or within 5% of a mean polyA sequence length in the plurality of chemically modified mRNA molecules.

Embodiment 60. The method of any one of Embodiments 49-59, wherein the polyA sequence length is measured by capillary gel electrophoresis (CGE) or by liquid chromatography (LC).

Embodiment 61. A method for producing a plurality of chemically modified mRNA molecules with polyA sequence lengths of at least about 200 consecutive adenosine nucleotides, comprising the steps of:
(a) in vitro transcribing the plurality of mRNA molecules in the presence of at least one chemically modified nucleotide, thereby producing a plurality of chemically modified mRNA molecules; and
(b) contacting the chemically modified mRNA molecules with a polyA polymerase under conditions to allow the synthesis of a polyA sequence to the 3′ end of the chemically modified mRNA molecules, thereby producing a plurality of chemically modified mRNA molecules with polyA sequence lengths of at least about 200 consecutive adenosine nucleotides;
wherein each mRNA molecule within the plurality of mRNA molecules comprise, from 5′ to 3′, a 5′ untranslated region (5′ UTR), at least one open reading frame (ORF), a 3′ untranslated region (3′ UTR), and a GC-rich sequence.

In order that this disclosure may be better understood, the following examples are set forth. These examples are for purposes of illustration only and are not to be construed as limiting the scope of the disclosure in any manner.

### EXAMPLES

### Example 1: Materials & Methods

mRNA Production
(1) In vitro transcription (IVT). mRNAs were produced as previously published (Kalnin et al (2021), NPJ Vaccines 6(1):61 and WO2021226436). Briefly, mRNAs were synthesized by in vitro transcription employing an RNA polymerase with a plasmid DNA template encoding the desired gene using 1 methyl-pseudo-UTP nucleotides to produce a modified mRNA. The IVT reaction was done in 50 ml conical tubes in an IVT buffer, with components including the template DNA, 100 mM ATP (Roche, cat. #04980824103), 100 mM GTP (Roche, cat. #04980859103), 100 mM CTP (Roche, cat. #04980875103), 100 mM UTP (Roche cat #04979818103), or 100 mM pseudo-UTP (Roche, cat #09188991103), SP6 Polymerase (Sigma, Cat #11487671001), RNase Inhibitor (Roche, Cat #3247058103) and pyrophosphatase (Aldevron, Cat #9132), at 37° C. for about 90 minutes. At the end of IVT reaction, DNase (Roche, Cat #3539121103) was added to remove the template DNA. Subsequently, the IVT product was purified with Qiagen RNeasy kit (cat #75162).
(2) Enzymatic Capping and Tailing Reactions. The resulting purified precursor mRNA was reacted further via enzymatic addition of a 5′ cap structure (Cap 1) and a 3′ poly (A) tail as determined by gel electrophoresis. (2a) Capping. Enzymatic capping was done in capping buffer with guanylyl transferase (Aldevron, Cat #9131) and 2′-O-methyl transferase (Aldevron, cat #9130) in the presence of GTP and SAM-tos (Sigma cat #A2408) and RNase Inhibitor, at 37° C. for 90 minutes. (2b) Tailing. At the end of capping reaction, 100 mM ATP, 10×polyA tailing buffer, and polyA polymerase (Aldevron cat #9133) were directly added to the tube. After 30 minutes incubation at 37° C., the reaction was stopped by EDTA (0.5M). mRNA material was purified again with Qiagen RNeasy kit and eluted in RNase free water.
   mRNA Analytics and Poly A Length Measurement

Concentration of the purified mRNA was measured by spectrophotometry on a Nanodrop (Thermal Fisher). mRNA integrity and the polyA tail length were measured by capillary gel electrophoresis (CGE) in a fragment analyzer (Agilent CGE system), using Agilent instruction manual (entitled, “5200, 5300, and 5400 Fragment Analyzer System Manual”, Document No: D0002110 Rev. A EDITION 02/2020, available for download on the website of Agilent: https://www.agilent.com/cs/library/usermanuals/public/Fragment_Analzyer_system_manual_D0002110.pdf). The length of polyA was calculated based on the mRNA size difference before and after polyA tailing.

### Example 2: GC Nucleotide Rich Sequences Enhance Tailing of Chemically Modified mRNA

The polyA tail plays an important role in mRNA stability and regulating translational efficiency. Enzymatic tailing of mRNA often produces polyA tails of varying lengths. The variable tail lengths in the composition of mRNA may contribute to variable stability and translational efficiency between individual mRNA molecules in the composition, which is undesirable in a pharmaceutical composition for therapy. This variability in tail length may be caused by a number of factors, including whether the mRNA is chemically modified (e.g., N1-methylpseudouridine modification).

With this in mind, polyA tail lengths were measured in enzymatically tailed N1-methylpseudouridine-modified mRNA under different conditions. Two DNA templates, codon optimized in different ways, were in vitro transcribed with either SP6 or T7. The generation of the DNA template with two different restriction enzymes (HindIII or SapI) for linearization was also tested. The tailing results were compared to an mRNA with an encoded polyA tail (i.e., the DNA template encoded the polyA tail, a non-enzymatic tailing method).

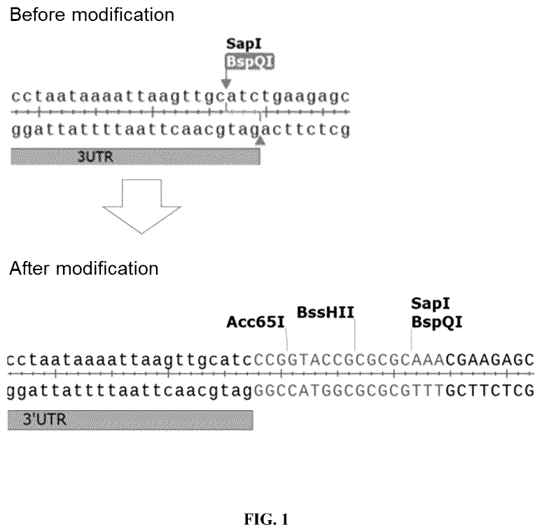

In an effort to maintain uniform tail lengths, the nucleotide sequence at the 3′ end of the 3′ UTR was altered to a more GC-rich sequence. Specifically, the sequence CCGGTACCGCGCGCAAAC (SEQ ID NO: 3) was inserted into the DNA template immediately after the 3′ UTR as shown in FIG. 1. This sequence, when inserted into the DNA template, is capable of being cleaved by the restriction enzymes BssHII, BspQI, and Acc651. The full sequence, with the BspQI binding site which sits outside of the cleavage site, is CCGGTACCGCGCGCAAACGAAGAGC (SEQ ID NO: 26). Cleavage of the DNA template with BssHII leaves the sequence CCGGTACCG, which when transcribed produces an untailed mRNA ending in the sequence CCGGUACCG (77.8% GC content). Cleavage of the DNA template with BspQI leaves the sequence CCGGTACCGCGCGC (SEQ ID NO: 2), which when transcribed produces an untailed mRNA ending in the sequence CCGGUACCGCGCGC (SEQ ID NO: 1) (87.5% GC content). Cleavage of the DNA template with Acc651 leaves the sequence CCG, which when transcribed produces an untailed mRNA ending in the sequence CCG (100% GC content).

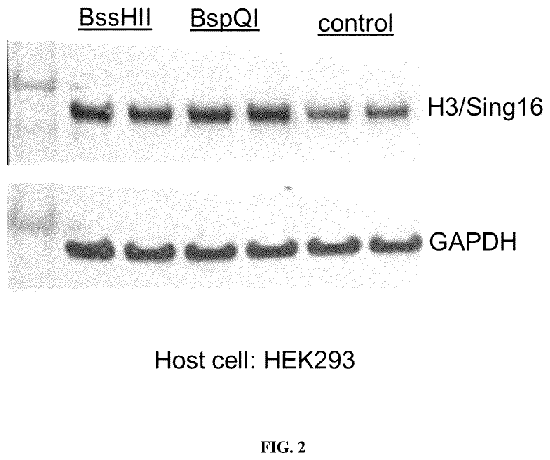

An unmodified DNA template (lacking the GC-rich sequence) led to a double-peak from a capillary gel electrophoresis (CGE) measurement, indicating a mixture of polyA tail lengths. However, when the GC-rich sequence was inserted, the subsequent linearized DNA template led to the production of mRNA with a more uniform polyA tail. Two different reactions with a BssHII-cut template and two different reactions with a BspQI-cut template produced a single peak in a CGE measurement, indicating a single species. The two BssHII-cut templates resulted in mRNA tail lengths of 336A and 488A and the two BspQI-cut templates resulted in mRNA tail lengths of 348A and 492A. The mRNA produced from these IVT and tailing reactions were transfected into HEK293 cells and the amount of the encoded polypeptide (influenza H3/Sing16) was detected by western blot. As shown in FIG. 2, the mRNA with the GC-rich sequences yielded better expression than the control mRNA lacking the GC-rich sequence.

To test whether the above recited GC-rich sequence worked in different mRNA contexts, the sequence was applied to a different mRNA encoding a different protein (influenza NA_B/Phuket13). The mRNA were produced via in vitro transcription from DNA templates linearized with the restriction enzyme BssHII or BspQI and in vitro transcribed with the RNA polymerase SP6 and polyA tailed with a polyA polymerase. An unmodified template in two separate reactions produced tailed mRNA with variable tail lengths and shorter tail lengths (about a 105 A residues) under the same reaction conditions. The CGE assay revealed a closely-spaced double peak indicating an earlier and later migrating species and the other reaction revealed a single peak indicating one species suggesting a lack of uniformity of tail lengths obtained under the same reaction conditions.

Differently, chemically modified mRNA produced from templates containing the GC-rich sequence yielded polyA tails that were longer (greater than 200 A residues) and more uniform across the pool of mRNA. This was observed with a BssHII-cut template (producing a tail length of 214A) and three separate experiments with a BspQI-cut template incubated at increasing time intervals with a polyA polymerase (producing tail lengths of 237A, 283A, and 379A, respectively), thereby yielding longer polyA tails.

A final alternative chemically modified mRNA was tested (encoding influenza NA/B-Colorado). For this mRNA, the template was linearized with only BspQI. The unmodified template yielded mRNA with non-uniform polyA tail lengths indicated by the double-peak suggestive of two species. The insertion of a GC-rich sequence yielded single peak measurements with consistently long and uniform polyA tails (with 330 A and 378 A tails, respectively).

### Example 3: Other GC Nucleotide Rich Sequences Also Enhance Tailing of Chemically Modified mRNA

The sequence CCGGTACCGCGCGCGTCGACGC (SEQ ID NO: 11) was inserted into the same DNA template as used in Example 2 immediately after the 3′ UTR. This sequence, when inserted into the DNA template, is capable of being cleaved by the restriction enzymes BssHII (as disclosed in Example 2), BspQI, Acc65I (as disclosed in Example 2), and SalI. Cleavage of the DNA template with BspQI leaves the sequence CCGGTACCGCGCGCGTCGA (SEQ ID NO: 12), which when transcribed produces an untailed mRNA ending in the sequence CCGGUACCGCGCGCGUCGA (SEQ ID NO: 13) (79% GC content). Cleavage of the DNA template with SalI leaves the sequence CCGGTACCGCGCGCG (SEQ ID NO: 14), which when transcribed produces an untailed mRNA ending in the sequence CCGGUACCGCGCGC (SEQ ID NO: 15) (86.7% GC content). The sequence CCGGUACCGCGCGC (SEQ ID NO: 15, SalI cleavage) was tested in similar conditions as in Example 2. The unmodified template yielded mRNA with non-uniform polyA tail lengths indicated by a double-peak suggestive of two species. In contrast, the insertion of the GC-rich sequence yielded single peak measurements consistent with long and uniform polyA tails.

The sequence CCGGTACCGCGCGCCTCGAGGC (SEQ ID NO: 16) was inserted into the same DNA template as used in Example 2 immediately after the 3′ UTR. This sequence, when inserted into the DNA template, is capable of being cleaved by the restriction enzymes BssHII (as disclosed in Example 2), BspQI, Acc65I (as disclosed in Example 2), and XhoI. Cleavage of the DNA template with BspQI leaves the sequence CCGGTACCGCGCGCCTCGA (SEQ ID NO: 17), which when transcribed produces an untailed mRNA ending in the sequence CCGGUACCGCGCGCCUCGA (SEQ ID NO: 18) (78.9% GC content). Cleavage of the DNA template with XhoI leaves the sequence CCGGTACCGCGCGCC (SEQ ID NO: 19), which when transcribed produces an untailed mRNA ending in the sequence CCGGUACCGCGCGCC (SEQ ID NO: 20) (86.7% GC content). The sequence CCGGUACCGCGCGCC (SEQ ID NO: 20, XhoI cleavage) was tested in similar conditions as in Example 2. The unmodified template yielded mRNA with non-uniform polyA tail lengths indicated by a double-peak suggestive of two species. In contrast, the insertion of the GC-rich sequence yielded single peak measurements consistent with long and uniform polyA tails.

The sequence CCGGTACCGCGCGCGGATCCGC (SEQ ID NO: 21) was inserted into the same DNA template as used in Example 2 immediately after the 3′ UTR. This sequence, when inserted into the DNA template, is capable of being cleaved by the restriction enzymes BssHII (as disclosed in Example 2), BspQI, Acc65I (as disclosed in Example 2), and BamHI. Cleavage of the DNA template with BspQI leaves the sequence CCGGTACCGCGCGCGGATC (SEQ ID NO: 22), which when transcribed produces an untailed mRNA ending in the sequence CCGGUACCGCGCGCGGAUC (SEQ ID NO: 23) (78.9% GC content). Cleavage of the DNA template with BamHI leaves the sequence CCGGTACCGCGCGCG (SEQ ID NO: 24), which when transcribed produces an untailed mRNA ending in the sequence CCGGUACCGCGCGCG (SEQ ID NO: 25) (86.7% GC content). The sequence CCGGUACCGCGCGCG (SEQ ID NO: 25, BamHI cleavage) was tested in similar conditions as in Example 2. The unmodified template yielded mRNA with non-uniform polyA tail lengths indicated by a double-peak suggestive of two species. In contrast, the insertion of the GC-rich sequence yielded single peak measurements consistent with long and uniform polyA tails.

The results described herein demonstrate that the inclusion of a GC-rich sequence at the 3′ end of the 3′ UTR (either in the 3′ UTR itself or immediately adjacent to it) yields polyA tailed mRNA of substantially uniform polyA tail length and long (greater than 200 A residues).

Other embodiments of the disclosure will be apparent to those skilled in the art from consideration of the specification and practice of the disclosure disclosed herein. It is intended that the specification and examples be considered as exemplary only, with a true scope of the disclosure being indicated by the following claims.

All patents and publications cited herein are incorporated by reference herein in their entirety.

1. A messenger RNA (mRNA) comprising, from 5′ to 3′, a 5′ untranslated region (5′ UTR), at least one open reading frame (ORF), a 3′ untranslated region (3′ UTR), and a GC-rich sequence which comprises at least about 75% G and/or C nucleotides and is at least 14 nucleotides in length, comprises CCGGUACCG, or comprises CCG, wherein the mRNA comprises at least one chemical modification.

2. The mRNA of claim 1, wherein
the GC-rich sequence comprises at least about 80% G and/or C nucleotides;
the GC-rich sequence comprises at least about 80% G and/or C nucleotides and is at least 14 nucleotides in length;
the GC-rich sequence comprises 100% G and/or C nucleotides;
the GC-rich sequence comprises CCGGUACCGCGCGC (SEQ ID NO: 1);
the GC-rich sequence comprises CCGGUACCGCGCGCGUCGA (SEQ ID NO: 13);
the GC-rich sequence comprises CCGGUACCGCGCGC (SEQ ID NO: 15);
the GC-rich sequence comprises CCGGUACCGCGCGCCUCGA (SEQ ID NO: 18);
the GC-rich sequence comprises CCGGUACCGCGCGCC (SEQ ID NO: 20);
the GC-rich sequence comprises CCGGUACCGCGCGCGGAUC (SEQ ID NO: 23);
the GC-rich sequence comprises CCGGUACCGCGCGCG (SEQ ID NO: 25);
the GC-rich sequence is contained within the 3′ UTR; and/or
the GC-rich sequence is not contained within the 3′ UTR.

3-13. (canceled)

14. The mRNA of claim 1, wherein
the chemical modification is pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, or 2′-O-methyl uridine;
the chemical modification is pseudouridine, N1-methylpseudouridine, 5-methylcytosine, 5-methoxyuridine, or a combination thereof; and/or
the chemical modification is N1-methylpseudouridine.

15-16. (canceled)

17. The mRNA of claim 1, further comprising
a polyA sequence;
the polyA sequence is present in the mRNA without enzymatic addition;
the polyA sequence is at least 10 consecutive adenosine nucleotides;
the polyA sequence is between 10 and 500 consecutive adenosine nucleotides;
the polyA sequence is between 80 and 300 consecutive adenosine nucleotides;
the mRNA contains a chimeric 5′ or 3′ UTR;
the mRNA encodes at least one polypeptide;
the polypeptide is a biologically active polypeptide, a therapeutic polypeptide, or an antigenic polypeptide;
the antigenic polypeptide is derived from a pathogen;
the polypeptide comprises an antibody or fragment thereof, enzyme replacement polypeptide, or genome-editing polypeptide;
the therapeutic polypeptide comprises an antibody heavy chain, an antibody light chain, an enzyme, or a cytokine;
the biologically active polypeptide comprises a genome-editing polypeptide;
the mRNA is synthesized using in vitro transcription (IVT); and/or
the mRNA is expressed in vivo or ex vivo.

18-30. (canceled)

31. A DNA polynucleotide comprising a nucleic acid sequence encoding the mRNA of claim 1.

32. A vector comprising the DNA polynucleotide of claim 31.

33. The vector of claim 32, wherein the vector comprises at least elements a-c, from 5′ to 3′:
a. an RNA polymerase promoter;
b. a polynucleotide sequence encoding an ORF; and
c. a polynucleotide sequence encoding a GC-rich sequence,
optionally wherein the vector comprises
d. a polynucleotide sequence encoding a restriction enzyme recognition site at least elements a-e, from 5′ to 3′:
a. an RNA polymerase promoter;
b. a polynucleotide sequence encoding a 5′ UTR;
c. a polynucleotide sequence encoding an ORF;
d. a polynucleotide sequence encoding a 3′ UTR;
e. a polynucleotide sequence encoding a GC-rich sequence;
f. a polynucleotide sequence encoding a restriction enzyme recognition site; and/or
g. a polynucleotide sequence encoding a polyadenylation signal,
and/or wherein
the vector lacks a polynucleotide sequence encoding a polyadenylation signal.

34-38. (canceled)

39. The vector of claim 32, wherein the vector comprises at least elements a-d, from 5′ to 3′:
a. an RNA polymerase promoter;
b. a polynucleotide sequence encoding a 5′ UTR;
c. a polynucleotide sequence encoding an ORF; and
d. a polynucleotide sequence encoding a 3′ UTR with a GC-rich sequence present at the 3′ end of the 3′UTR, optionally wherein the vector further comprises:
e. a polynucleotide sequence encoding a restriction enzyme recognition site;
f. a polynucleotide sequence encoding a polyadenylation signal; and/or
wherein the vector lacks a polynucleotide sequence encoding a polyadenylation signal

40-42. (canceled)

43. The vector of claim 33, wherein
the restriction enzyme recognition site comprises one or more of a BspQI recognition site, a BssHII recognition site, a SalI recognition site, a XhoI recognition site, a BamHI recognition site, and a Acc65I recognition site;
the polynucleotide sequence encoding a GC-rich sequence comprises
(SEQ ID NO: 3)
CCGGTACCGCGCGCAAAC;
the polynucleotide sequence encoding a GC-rich sequence comprises
(SEQ ID NO: 26)
CCGGTACCGCGCGCAAACGAAGAGC;
the polynucleotide sequence encoding a GC-rich sequence comprises
(SEQ ID NO: 11)
CCGGTACCGCGCGCGTCGACGC;
the polynucleotide sequence encoding a GC-rich sequence comprises
(SEQ ID NO: 12)
CCGGTACCGCGCGCGTCGA;
the polynucleotide sequence encoding a GC-rich sequence comprises
(SEQ ID NO: 14)
CCGGTACCGCGCGCG;
the polynucleotide sequence encoding a GC-rich sequence comprises
(SEQ ID NO: 16)
CCGGTACCGCGCGCCTCGAGGC;
the polynucleotide sequence encoding a GC-rich sequence comprises
(SEQ ID NO: 17)
CCGGTACCGCGCGCCTCGA;
the polynucleotide sequence encoding a GC-rich sequence comprises
(SEQ ID NO: 19)
CCGGTACCGCGCGCC;
the polynucleotide sequence encoding a GC-rich sequence comprises
(SEQ ID NO: 21)
CCGGTACCGCGCGCGGATCCGC;
the polynucleotide sequence encoding a GC-rich sequence comprises
(SEQ ID NO: 22)
CCGGTACCGCGCGCGGATC;
the polynucleotide sequence encoding a GC-rich sequence comprises
(SEQ ID NO: 24)
CCGGTACCGCGCGCG.

44-54. (canceled)

55. A host cell comprising the vector of claim 32.

56. A pharmaceutical composition comprising the mRNA of claim 1.

57. A vector comprising the DNA polynucleotide of claim 31, wherein the vector comprises
at least elements a-d, from 5′ to 3′:
a. an RNA polymerase promoter;
b. a polynucleotide sequence encoding an ORF;
c. a polynucleotide sequence encoding a GC-rich sequence; and
d. a polynucleotide sequence encoding a restriction enzyme recognition site, wherein the restriction enzyme recognition site comprises one or more of a BspQI recognition site, a BssHII recognition site, a SalI recognition site, a XhoI recognition site, a BamHI recognition site, and a Acc65I recognition site; optionally wherein
the vector comprises at least elements a-f, from 5′ to 3′:
a. an RNA polymerase promoter;
b. a polynucleotide sequence encoding a 5′ UTR;
c. a polynucleotide sequence encoding an ORF;
d. a polynucleotide sequence encoding a 3′ UTR; and
e. a polynucleotide sequence encoding a GC-rich sequence;
f. a polynucleotide sequence encoding a restriction enzyme recognition site, wherein the restriction enzyme recognition site comprises one or more of a BspQI recognition site, a BssHII recognition site, a SalI recognition site, a XhoI recognition site, a BamHI recognition site, and a Acc65I recognition site.

58. (canceled)

59. The vector of claim 57, further comprising
a polynucleotide sequence encoding a polyadenylation signal;
the vector lacks a polynucleotide sequence encoding a polyadenylation signal;
the polynucleotide sequence encoding a GC-rich sequence comprises
(SEQ ID NO: 3)
CCGGTACCGCGCGCAAAC;
the polynucleotide sequence encoding a GC-rich sequence comprises
(SEQ ID NO: 26)
CCGGTACCGCGCGCAAACGAAGAGC;
the polynucleotide sequence encoding a GC-rich sequence comprises
(SEQ ID NO: 11)
CCGGTACCGCGCGCGTCGACGC;
the polynucleotide sequence encoding a GC-rich sequence comprises
(SEQ ID NO: 12)
CCGGTACCGCGCGCGTCGA;
the polynucleotide sequence encoding a GC-rich sequence comprises
(SEQ ID NO: 14)
CCGGTACCGCGCGCG;
the polynucleotide sequence encoding a GC-rich sequence comprises
(SEQ ID NO: 16)
CCGGTACCGCGCGCCTCGAGGC;
the polynucleotide sequence encoding a GC-rich sequence comprises
(SEQ ID NO: 17)
CCGGTACCGCGCGCCTCGA;
the polynucleotide sequence encoding a GC-rich sequence comprises
(SEQ ID NO: 19)
CCGGTACCGCGCGCC;
the polynucleotide sequence encoding a GC-rich sequence comprises
(SEQ ID NO: 21)
CCGGTACCGCGCGCGGATCCGC;
the polynucleotide sequence encoding a GC-rich sequence comprises
(SEQ ID NO: 22)
CCGGTACCGCGCGCGGATC;
the polynucleotide sequence encoding a GC-rich sequence comprises
(SEQ ID NO: 24)
CCGGTACCGCGCGCG.

60-72. (canceled)

73. A method for producing a plurality of chemically modified mRNA molecules with similar polyA sequence lengths, comprising the steps of:
(a) in vitro transcribing the plurality of mRNA molecules in the presence of at least one chemically modified nucleotide, thereby producing a plurality of chemically modified mRNA molecules; and
(b) contacting the chemically modified mRNA molecules with a polyA polymerase under conditions to allow the synthesis of a polyA sequence to the 3′ end of the chemically modified mRNA molecules, thereby producing a plurality of chemically modified mRNA molecules with similar polyA sequence lengths;
wherein each mRNA molecule within the plurality of mRNA molecules comprise, from 5′ to 3′, a 5′ untranslated region (5′ UTR), at least one open reading frame (ORF), a 3′ untranslated region (3′ UTR), and a GC-rich sequence.

74. The method of claim 73, wherein
the GC-rich sequence is contained within the 3′ UTR; and/or
the GC-rich sequence is not contained within the 3′ UTR.

75. (canceled)

76. The method of claim 73, wherein
the presence of the GC-rich sequence in each mRNA molecule within the plurality of mRNA molecules facilitates the generation of polyA sequences of substantially the same length;
at least 60% of the chemically modified mRNA molecules within the plurality of chemically modified mRNA molecules comprise a polyA sequence length that is substantially the same;
about 60%, about 70%, about 80%, about 85% m, about 90%, about 95%, or about 99% of the chemically modified mRNA molecules within the plurality of chemically modified mRNA molecules comprise a polyA sequence length that is substantially the same;
substantially all of the chemically modified mRNA molecules within the plurality of chemically modified mRNA molecules comprise a polyA sequence length that is substantially the same;
at least 60% of the chemically modified mRNA molecules within the plurality of chemically modified mRNA molecules comprise a polyA sequence length of about 50 to about 100 consecutive adenosine nucleotides, about 100 to about 150 consecutive adenosine nucleotides, about 150 to about 200 consecutive adenosine nucleotides, about 200 to about 250 consecutive adenosine nucleotides, about 250 to about 300 consecutive adenosine nucleotides, about 300 to about 350 consecutive adenosine nucleotides, about 350 to about 400 consecutive adenosine nucleotides, about 400 to about 450 consecutive adenosine nucleotides, or about 450 to about 500 consecutive adenosine nucleotides;
about 60%, about 70%, about 80%, about 85%, about 90%, about 95%, or about 99% of the chemically modified mRNA molecules within the plurality of chemically modified mRNA molecules comprise a polyA sequence length of about 50 to about 100 consecutive adenosine nucleotides, about 100 to about 150 consecutive adenosine nucleotides, about 150 to about 200 consecutive adenosine nucleotides, about 200 to about 250 consecutive adenosine nucleotides, about 250 to about 300 consecutive adenosine nucleotides, about 300 to about 350 consecutive adenosine nucleotides, about 350 to about 400 consecutive adenosine nucleotides, about 400 to about 450 consecutive adenosine nucleotides, or about 450 to about 500 consecutive adenosine nucleotides;
substantially all of the chemically modified mRNA molecules within the plurality of chemically modified mRNA molecules comprise a polyA sequence length of about 50 to about 100 consecutive adenosine nucleotides, about 100 to about 150 consecutive adenosine nucleotides, about 150 to about 200 consecutive adenosine nucleotides, about 200 to about 250 consecutive adenosine nucleotides, about 250 to about 300 consecutive adenosine nucleotides, about 300 to about 350 consecutive adenosine nucleotides, about 350 to about 400 consecutive adenosine nucleotides, about 400 to about 450 consecutive adenosine nucleotides, or about 450 to about 500 consecutive adenosine nucleotides;
the polyA sequence lengths in the plurality of chemically modified mRNA molecules comprise a polyA sequence length that is within 50%, within 45%, within 40%, within 35%, within 30%, within 25%, within 20%, within 15%, within 10%, or within 5% of a mean polyA sequence length in the plurality of chemically modified mRNA molecules; and/or
the polyA sequence length is measured by capillary gel electrophoresis (CGE) or by liquid chromatography (LC).

77-84. (canceled)

85. The method of claim 73, wherein
the GC-rich sequence comprises at least about 50% G and/or C nucleotides to 100% G and/or C nucleotides;
the GC-rich sequence comprises at least about 70% G and/or C nucleotides;
the GC-rich sequence comprises at least about 80% G and/or C nucleotides;
the GC-rich sequence comprises at least about 80% G and/or C nucleotides and is at least 14 nucleotides in length; and/or
the GC-rich sequence comprises 100% G and/or C nucleotides.

86-89. (canceled)

90. The method of claim 73, wherein
the GC-rich sequence comprises CCGGUACCG;
wherein the GC-rich sequence comprises CCGGUACCGCGCGC (SEQ ID NO: 1);
the GC-rich sequence comprises CCGGUACCGCGCGCGUCGA (SEQ ID NO: 13);
the GC-rich sequence comprises CCGGUACCGCGCGC (SEQ ID NO: 15);
the GC-rich sequence comprises CCGGUACCGCGCGCCUCGA (SEQ ID NO: 18);
the GC-rich sequence comprises CCGGUACCGCGCGCC (SEQ ID NO: 20);
the GC-rich sequence comprises CCGGUACCGCGCGCGGAUC (SEQ ID NO: 23);
the GC-rich sequence comprises CCGGUACCGCGCGCG (SEQ ID NO: 25); and/or
the GC-rich sequence comprises CCG.

91-98. (canceled)

99. A method for producing a plurality of chemically modified mRNA molecules with polyA sequence lengths of at least about 50 consecutive adenosine nucleotides, comprising the steps of:
(a) in vitro transcribing the plurality of mRNA molecules in the presence of at least one chemically modified nucleotide, thereby producing a plurality of chemically modified mRNA molecules; and
(b) contacting the chemically modified mRNA molecules with a polyA polymerase under conditions to allow the synthesis of a polyA sequence to the 3′ end of the chemically modified mRNA molecules, thereby producing a plurality of chemically modified mRNA molecules with polyA sequence lengths of at least about 200 consecutive adenosine nucleotides;
wherein each mRNA molecule within the plurality of mRNA molecules comprise, from 5′ to 3′, a 5′ untranslated region (5′ UTR), at least one open reading frame (ORF), a 3′ untranslated region (3′ UTR), and a GC-rich sequence.

100. The method of claim 99, wherein
the GC-rich sequence is contained within the 3′ UTR;
the GC-rich sequence is not contained within the 3′ UTR;
the presence of the GC-rich sequence in each mRNA molecule within the plurality of mRNA molecules facilitates the generation of polyA sequences of substantially the same length of at least about 50 consecutive adenosine nucleotides;
at least 60% of the chemically modified mRNA molecules within the plurality of chemically modified mRNA molecules comprise a polyA sequence length that is substantially the same of at least about 50 consecutive adenosine nucleotides;
about 60%, about 70%, about 80%, about 85%, about 90%, about 95%, or about 99% of the chemically modified mRNA molecules within the plurality of chemically modified mRNA molecules comprise a polyA sequence length that is substantially the same of at least about 50 consecutive adenosine nucleotides;
substantially all of the chemically modified mRNA molecules within the plurality of chemically modified mRNA molecules comprise a polyA sequence length that is substantially the same of at least about 50 consecutive adenosine nucleotides;
at least 60% of the chemically modified mRNA molecules within the plurality of chemically modified mRNA molecules comprise a polyA sequence length of about 50 to about 100 consecutive adenosine nucleotides, about 100 to about 150 consecutive adenosine nucleotides, about 150 to about 200 consecutive adenosine nucleotides, about 200 to about 250 consecutive adenosine nucleotides, about 250 to about 300 consecutive adenosine nucleotides, about 300 to about 350 consecutive adenosine nucleotides, about 350 to about 400 consecutive adenosine nucleotides, about 400 to about 450 consecutive adenosine nucleotides, or about 450 to about 500 consecutive adenosine nucleotides;
at least 60% of the chemically modified mRNA molecules within the plurality of chemically modified mRNA molecules comprise a polyA sequence length of about 50, about 80, about 100, about 120, about 150, about 180, about 200, about 220, about 250, about 280, about 300, about 320, about 350, about 380, about 400, about 420, about 450, about 480, or about 500 consecutive adenosine nucleotides;
about 60%, about 70%, about 80%, about 85%, about 90%, about 95%, or about 99% of the chemically modified mRNA molecules within the plurality of chemically modified mRNA molecules comprise a polyA sequence length of about 50 to about 100 consecutive adenosine nucleotides, about 100 to about 150 consecutive adenosine nucleotides, about 150 to about 200 consecutive adenosine nucleotides, about 200 to about 250 consecutive adenosine nucleotides, about 250 to about 300 consecutive adenosine nucleotides, about 300 to about 350 consecutive adenosine nucleotides, about 350 to about 400 consecutive adenosine nucleotides, about 400 to about 450 consecutive adenosine nucleotides, or about 450 to about 500 consecutive adenosine nucleotides;
about 60%, about 70%, about 80%, about 85%, about 90%, about 95%, or about 99% of the chemically modified mRNA molecules within the plurality of chemically modified mRNA molecules comprise a polyA sequence length of about 50, about 80, about 100, about 120, about 150, about 180, about 200, about 220, about 250, about 280, about 300, about 320, about 350, about 380, about 400, about 420, about 450, about 480, or about 500 consecutive adenosine nucleotides
substantially all of the chemically modified mRNA molecules within the plurality of chemically modified mRNA molecules comprise a polyA sequence length of about 50 to about 100 consecutive adenosine nucleotides, about 100 to about 150 consecutive adenosine nucleotides, about 150 to about 200 consecutive adenosine nucleotides, about 200 to about 250 consecutive adenosine nucleotides, about 250 to about 300 consecutive adenosine nucleotides, about 300 to about 350 consecutive adenosine nucleotides, about 350 to about 400 consecutive adenosine nucleotides, about 400 to about 450 consecutive adenosine nucleotides, or about 450 to about 500 consecutive adenosine nucleotides;
substantially all of the chemically modified mRNA molecules within the plurality of chemically modified mRNA molecules comprise a polyA sequence length of about 50, about 80, about 100, about 120, about 150, about 180, about 200, about 220, about 250, about 280, about 300, about 320, about 350, about 380, about 400, about 420, about 450, about 480, or about 500 consecutive adenosine nucleotides;
the polyA sequence lengths in the plurality of chemically modified mRNA molecules comprise a polyA sequence length that is within 50%, within 45%, within 40%, within 35%, within 30%, within 25%, within 20%, within 15%, within 10%, or within 5% of a mean polyA sequence length in the plurality of chemically modified mRNA molecules; and/or
the polyA sequence length is measured by capillary gel electrophoresis (CGE) or by liquid chromatography (LC).

101-113. (canceled)


SEQUENCE LISTING (26 sequences)

SEQ ID NO: 1 - RNA, 14 nt, synthetic construct
  source 1..14: mol_type = other RNA
ccggtaccgcgcgc

SEQ ID NO: 2 - DNA, 14 bp, synthetic construct
  source 1..14: mol_type = other DNA
ccggtaccgcgcgc

SEQ ID NO: 3 - DNA, 18 bp, synthetic construct
  source 1..18: mol_type = other DNA
ccggtaccgcgcgcaaac

SEQ ID NO: 4 - DNA, 30 bp, synthetic construct
  source 1..30: mol_type = other DNA
cctaataaaattaagttgcatctgaagagc

SEQ ID NO: 5 - DNA, 30 bp, synthetic construct
  source 1..30: mol_type = other DNA
gctcttcagatgcaacttaattttattagg

SEQ ID NO: 6 - DNA, 44 bp, synthetic construct
  source 1..44: mol_type = other DNA
cctaataaaattaagcatcccggtaccgcgcgcaaacgaagagc

SEQ ID NO: 7 - DNA, 47 bp, synthetic construct
  source 1..47: mol_type = other DNA
gctcttcgtttgcgcgcggtaccgggatgcaacttaattttattagg

SEQ ID NO: 8 - RNA, 11 nt, synthetic construct
  source 1..11: mol_type = other RNA
gggatcctacc

SEQ ID NO: 9 - RNA, 140 nt, synthetic construct
  source 1..140: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacg

SEQ ID NO: 10 - RNA, 100 nt, synthetic construct
  source 1..100: mol_type = other RNA
cgggtggcatccctgtgacccctccccagtgcctctcctggccctggaagttgccactccagtgcccaccagccttgtcctaataaaattaagttgcatc

SEQ ID NO: 11 - DNA, 22 bp, synthetic construct
  source 1..22: mol_type = other DNA
ccggtaccgcgcgcgtcgacgc

SEQ ID NO: 12 - DNA, 19 bp, synthetic construct
  source 1..19: mol_type = other DNA
ccggtaccgcgcgcgtcga

SEQ ID NO: 13 - RNA, 19 nt, synthetic construct
  source 1..19: mol_type = other RNA
ccggtaccgcgcgcgtcga

SEQ ID NO: 14 - DNA, 15 bp, synthetic construct
  source 1..15: mol_type = other DNA
ccggtaccgcgcgcg

SEQ ID NO: 15 - RNA, 14 nt, synthetic construct
  source 1..14: mol_type = other RNA
ccggtaccgcgcgc

SEQ ID NO: 16 - DNA, 22 bp, synthetic construct
  source 1..22: mol_type = other DNA
ccggtaccgcgcgcctcgaggc

SEQ ID NO: 17 - DNA, 19 bp, synthetic construct
  source 1..19: mol_type = other DNA
ccggtaccgcgcgcctcga

SEQ ID NO: 18 - RNA, 19 nt, synthetic construct
  source 1..19: mol_type = other RNA
ccggtaccgcgcgcctcga

SEQ ID NO: 19 - DNA, 15 bp, synthetic construct
  source 1..15: mol_type = other DNA
ccggtaccgcgcgcc

SEQ ID NO: 20 - RNA, 15 nt, synthetic construct
  source 1..15: mol_type = other RNA
ccggtaccgcgcgcc

SEQ ID NO: 21 - DNA, 22 bp, synthetic construct
  source 1..22: mol_type = other DNA
ccggtaccgcgcgcggatccgc

SEQ ID NO: 22 - DNA, 19 bp, synthetic construct
  source 1..19: mol_type = other DNA
ccggtaccgcgcgcggatc

SEQ ID NO: 23 - RNA, 19 nt, synthetic construct
  source 1..19: mol_type = other RNA
ccggtaccgcgcgcggatc

SEQ ID NO: 24 - DNA, 15 bp, synthetic construct
  source 1..15: mol_type = other DNA
ccggtaccgcgcgcg

SEQ ID NO: 25 - RNA, 15 nt, synthetic construct
  source 1..15: mol_type = other RNA
ccggtaccgcgcgcg

SEQ ID NO: 26 - DNA, 25 bp, synthetic construct
  source 1..25: mol_type = other DNA
ccggtaccgcgcgcaaacgaagagc